(19) United States
(12) Patent Application Publication
(10) Pub. No.: US 2019/0008941 A1
(43) Pub. Date: Jan. 10, 2019

(54) COMBINED THERAPY AND PROPHYLAXIS FOR GENITAL TRACT INFECTIONS

(71) Applicant: The Research Foundation for The State University of New York, Buffalo, NY (US)

(72) Inventors: Michael W. RUSSELL, East Amherst, NY (US); Yingru LIU, Williamsville, NY (US); Nejat K. EGILMEZ, Louisville, KY (US)

(21) Appl. No.: 16/138,526

(22) Filed: Sep. 21, 2018

Related U.S. Application Data
(63) Continuation-in-part of application No. 15/824,700, filed on Nov. 28, 2017.
Application No. 15/824,700 is a continuation-in-part of application No. 14/404,197, filed on Nov. 26, 2014, now Pat. No. 9,827,319.
(60) Provisional application No. 61/652,630, filed on May 29, 2012.

(51) Int. Cl. A61K 39/02; A61K 39/39

(52) U.S. Cl. CPC A61K 39/0208; A61K 2039/545; A61K 39/39

(57) ABSTRACT

Provided is a method for treating and reducing the recurrence of genital tract infections such as gonococcal infections. The method comprises local application of IL-12 incorporated in polymeric microspheres. A method is also provided to reduce the incidence of genital tract infections caused by N. gonorrhoeae by administration of outer membrane vesicle preparations from N. gonorrhoeae and IL-12 incorporated in polymeric microspheres.

### CROSS-REFERENCE TO RELATED APPLICATIONS

This application is a continuation-in-part of U.S. patent application Ser. No. 15/824,700, filed on Nov. 28, 2017, which is a continuation-in-part of U.S. patent application Ser. No. 14/404,197, filed on Nov. 26, 2014, now U.S. Pat. No. 9,827,319, which is a National Phase of International Patent Application No. PCT/US13/43068, filed May 29, 2013, which in turn claims priority to U.S. Provisional Application No. 61/652,630, filed on May 29, 2012, the disclosures of all of which are incorporated herein by reference.

### STATEMENT REGARDING FEDERALLY SPONSORED RESEARCH

This invention was made with government support under grant AI074791 awarded by the National Institutes of Health. The government has certain rights in the invention.

### FIELD OF THE INVENTION

The invention relates to compositions comprising IL-12 and outer membrane vesicles from Neisseria gonorrhoeae, and methods for using such compositions for treatment of genital tract infections.

### BACKGROUND OF THE INVENTION

Genital tract infection by Neisseria gonorrhoeae gives rise to gonorrhea, which is the second most frequent reportable infectious disease in the US affecting >300,000 individuals per annum, although the real incidence is believed to be at least double that number. The worldwide incidence of gonorrhea is estimated to be >100 million cases per year. Women bear the brunt of the infection, because untreated gonorrhea can ascend into the upper reproductive tract and give rise to pelvic inflammatory disease and tubal scarring, leading to infertility and risk for ectopic pregnancy which can be life-threatening. Yet a large proportion of infected women, variously given as up to 50% or even more, can be asymptomatically infected, thereby increasing the risk of spreading the infection among their sexual contacts. Men by contrast usually become aware of their infection within a few days and are therefore impelled to seek treatment. New-born infants can become infected in the eyes as a result of delivery through an infected birth canal, and this can lead to blindness if left untreated. Untreated gonorrhea is also known to increase the risk for acquiring and transmitting HIV up to 5-fold. Treatment depends upon antibiotics, but N. gonorrhoeae has quickly become resistant to each class of antibiotics used against it, including most recently the fluorquinolones (ciprofloxacin), and the currently recommended antibiotics are cephalosporins. However, resistance to these has begun to emerge, making N. gonorrhoeae multiple-drug-resistant. Despite various efforts, no vaccine against N. gonorrhoeae is currently available. Vaccine efforts are complicated by the extensive antigenic variability of N. gonorrhoeae, in which most major surface antigens, including lipooligosaccharide (LOS), porin, pilin, and the opacity proteins (Opa) are subject to phase-variable expression (LOS, Opa, pilus), allelic variation (porin, Opa), or recombinatorial expression (pilin). Thus options for treatment and control of the disease are becoming limited. A puzzling but well-known feature of gonorrhea is that recovery from infection does not lead to protective immunity against re-infection, and repeated infections are common.

### SUMMARY OF THE INVENTION

This disclosure provides a method of reducing the risk of developing N. gonorrhoeae infections by administering to an individual N. gonorrhoeae antigens and IL-12 incorporated in polymeric microspheres. The N. gonorrhoeae antigens may be in the form of outer membrane vesicles or microvesicles. Other forms of antigens (such as purified or semi-purified) may also be used. The N. gonorrhoeae antigen preparation (such as OMVs) and the IL-12 microspheres may be delivered in a single composition or different compositions, by the same route or different routes, at the same time or different times, over a same time period and delivery regimen or different time period and delivery regimens. For example, the N. gonorrhoeae OMVs and IL-12 microspheres can be delivered intravaginally, or may be delivered intranasally.

In one aspect, this disclosure provides a method for treatment of cervico-vaginal infections by local application of IL-12 incorporated in polymeric microspheres. While not intending to be bound by any particular theory, it is considered that application of IL-12 incorporated in polymeric microspheres locally to mucosal surfaces enhances the body's own immune response against an existing infection resulting in reduction or elimination of that infection and/or generation of immunity against repeat infection. In one embodiment, the amount is sufficient to promote Th1-driven response against the microorganisms causing the infection. The amount of IL-12 may be sufficient to provide a therapeutic effect, a prophylactic effect, or both against the causative microorganisms. Infections that can be treated by the present method include, but are not limited to, those that are caused by N. gonorrhoeae, C. trachomatis or both. An example of a polymer that can be used for microencapsulation of IL-12 is polylactic acid.

In one aspect, this disclosure provides a composition comprising OMVs prepared from N. gonorrhoeae and IL-12 containing microspheres (ms) suitable for intravaginal delivery.

In one aspect, this disclosure provides a kit for intravaginal delivery of OMVs prepared from N. gonorrhoeae and IL-12 containing microspheres. The OMVs and IL-12 ms may be present as separate compositions or the same composition. The kit may comprise multiple doses of the OMV composition and the IL-12 composition and instructions for administration, which may include instruction on frequency, length of administration regimen, mode of administration and the like.

### BRIEF DESCRIPTION OF THE DRAWINGS

For a fuller understanding of the nature and objects of the invention, reference should be made to the following detailed description taken in conjunction with the accompanying drawings, in which:

FIG. 1 is a graph showing the effects of intravaginal treatment with 1 μg of IL-12 encapsulated in polylactic acid (PLA) microspheres on the course of vaginal infection with N. gonorrhoeae in mice;

FIG. 2 is a graph showing the effects of intravaginal treatment with IL-12 microspheres during primary infection with N. gonorrhoeae (FIG. 1) on the course of secondary vaginal infection with N. gonorrhoeae in mice.

FIG. 3 is a graph showing the effects of intravaginal treatment with 1 μg of soluble vs. microencapsulated IL-12 on the course of vaginal infection with N. gonorrhoeae in mice.

FIG. 4 is a graph showing the effect of intravaginal IL-12 microsphere (ms) treatment on primary gonococcal infection in BALB/c mice. (A) IL-12 ms dose optimization experiment. Microspheres containing the stated doses of IL-12 were given on days 0, 2, 4, 6, 8; n=8 mice per group. N. gonorrhoeae (Ngo) burden was monitored daily by vaginal swab culture. Significant differences in infection burdens were found between mice treated with 2.0 μg (p<0.01), 1.0 μg (p<0.01), or 0.5 μg (p<0.05) of microencapsulated IL-12 and controls (ANOVA). (B) Time course of infection in mice treated with IL-12 ms, soluble IL-12, IL-17 ms, or control ms, or in untreated mice; cytokine dose=1.0 μg given on days −1, 1, 3, 5, 7; n=8 mice per group. Significant differences in infection burdens were found between mice treated with IL-12 ms (p<0.01) or IL-17 ms (p ˜0.01) and controls (ANOVA). (C) Data from the experiment shown in B plotted as percentage of mice remaining infected under the indicated cytokine treatments. Infection was cleared significantly faster in mice treated with IL-12 ms (p<0.0001) or IL-17 ms (p<0.001) than in controls (Kaplan-Meier). (D) Cytokine expression in isolated ILN cells from sham-infected or infected mice with IL-12 ms, IL-17 ms, or control ms treatment; n=7 mice per group. Expression of IFN-γ, IL-4, and IL-17 in CD4⁺ T cells isolated at day 5 after infection was analyzed by flow cytometry. (E) RT-PCR analysis of IFN-γ, IL-4, and IL-17 mRNA levels in vaginal tissue harvested at day 3 from sham-infected or infected mice with IL-12 ms, IL-17 ms, or control ms treatment; n=7 mice per group. Cytokine gene expression levels detected by RT-PCR were normalized relative to expression of β-actin and set at 1.0 for sham-infected group. (F) Phenotypic profile of vaginal cells isolated on day 5 from sham-infected or infected mice treated with IL-17 ms or control ms; n=7 mice per group. (G) Vaginal and (H) serum anti-gonococcal IgA and IgG antibody responses in sham-infected or infected mice with IL-12 ms, IL-17 ms, or control ms treatment; n=7 mice per group. Vaginal washes and sera were collected 15 days after inoculation, and gonococcus-specific and total IgA and IgG were measured by ELISA. Results from one representative out of three independent experiments are shown. In D-H, #p<0.05; *p<0.01 (unpaired t test);

FIG. 5 is a graph showing the effect of intravaginal IL-12 microsphere (ms) treatment during primary infection on secondary gonococcal infection. (A) Time course of secondary infection in mice treated with IL-12 ms, soluble IL-12, IL-17 ms, or control ms during primary infection, or in previously sham-infected mice with or without IL-12 ms treatment; n=8 mice per group. Significant differences in infection burdens were found between mice previously treated with IL-12 ms (p ˜0.01) and controls (ANOVA). (B) Data from the experiment shown in A plotted as percentage of mice remaining infected after reinfection under the indicated treatments during primary infection. Infection was cleared significantly faster in mice previously treated with IL-12 ms (p<0.0001) than in controls (Kaplan-Meier). (C) Flow cytometric analysis of cytokine expression in ILN CD4⁺ T cells isolated at day 5 from reinfected mice treated with IL-12 ms, IL-17 ms, or control ms during primary infection, or from mice that were sham-infected in both primary and secondary phases (“sham-reinfected”); n=7 mice per group. (D) RT-PCR analysis of IFN-γ, IL-4, and IL-17 mRNA levels in vaginas harvested at day 3 from sham-reinfected or reinfected mice treated with IL-12 ms, IL-17 ms, or blank ms during primary infection; n=7 mice per group. Cytokine gene expression levels detected by RT-PCR were normalized relative to expression of β-actin and set at 1.0 for sham-reinfected group. (E) Vaginal and (F) serum anti-gonococcal IgA and IgG antibody responses to secondary infection in sham-reinfected or reinfected mice treated with IL-12 ms, IL-17 ms, or blank ms during primary infection; n=7 mice per group. Vaginal washes and sera were collected 15 days after inoculation, and gonococcus-specific and total IgA and IgG were measured by ELISA. Results from one representative out of three independent experiments are shown. In C-F, #p<0.05; *p<0.01 (unpaired t test).

FIG. 6 shows intravaginal (I.vag) immunization with gonococcal OMV plus IL-12/ms induced resistance to genital infection with N. gonorrhoeae, and generated an immune response. a: Mice were immunized 3 times at 7-day intervals with OMV (40 μg protein) from strain FA1090 plus control (blank) ms or IL-12/ms (1 μg IL-12); control mice were sham-immunized with either blank ms, or with IL-12/ms alone. Two weeks after the last immunization, all mice were challenged by i.vag. inoculation with N. gonorrhoeae strain FA1090 (5×10⁶ CFU), and infection was monitored by vaginal swabbing and plating. Left panel: recovery (CFU) of N. gonorrhoeae (mean±SEM, N=8 mice), *P<0.01 (ANOVA); right panel: % of animals remaining infected at each time point, P<0.01 (Kaplan-Meier analysis, log-rank test, OMV plus IL-12/ms vs. OMV plus blank ms). b: Vaginal wash (left) and serum (right) antibodies against strain FA1090 in samples collected after termination (day 15), shown as mean±SEM, N=5 samples; #P<0.05, *P<0.01, Student's t. c: Intracellular cytokine staining in CD4⁺ cells recovered from ILN at termination (day 15), shown as mean±SEM, N=3 samples, % of CD4⁺ staining for each cytokine; *P<0.01 Student's t. d: Mice were immunized twice at a 14-day interval with gonococcal (Ngo) OMV (40 μg protein) plus blank ms or IL-12/ms (1 μg IL-12); control mice were sham-immunized with blank ms alone or with NTHI OMV (40 μg protein) plus IL-12/ms (1 μg IL-12). Two weeks later, all mice were challenged with N. gonorrhoeae FA1090 (5×10⁶ CFU). Left panel: recovery (CFU) of N. gonorrhoeae (mean±SEM, N=8 mice), *P<0.01 (ANOVA, gonococcal OMV plus IL-12/ms vs. gonococcal OMV plus blank ms); right panel: % of animals remaining infected at each time point, P<0.0001 (Kaplan-Meier analysis, log-rank test, gonococcal OMV plus IL-12/ms vs. gonococcal OMV plus blank ms).

FIG. 7 shows antibody responses generated by immunization with gonococcal OMV plus IL-12/ms, prior to gonococcal challenge. a: Vaginal wash (left panel) and serum (right panel) antibodies assayed by ELISA 2 weeks after the last immunization with 1, 2, or 3 doses of gonococcal OMV (40 μg protein) plus IL-12/ms (1 μg IL-12). Control samples were obtained from mice sham-immunized with blank ms (3 doses); additional mice were immunized 3× with gonococcal OMV plus blank ms. Data shown as mean±SEM, N=5 samples, #P<0.05, *P<0.01 relative to control samples (ANOVA). Duration of vaginal wash (b) and serum (c) antibodies in mice immunized with 2 doses of FA1090 OMV plus blank ms or IL-12/ms; data shown as mean±SEM, N=5 samples; C, control samples from unimmunized mice.

FIG. 8 shows: a: T cell cytokine responses in ILN cells induced by immunization with gonococcal OMV plus IL-12/ms 2 weeks after the last immunization with 1, 2, or 3 immunizations with gonococcal OMV (40 μg protein) plus IL-12/ms (1 μg IL-12). Control ILN were obtained from mice sham-immunized with blank ms (3 doses) and additional mice were immunized 3× with gonococcal OMV plus blank ms. Data shown as mean±SEM, N=3 samples, % of CD4⁺ or CD8⁺ cells staining for each cytokine. *P<0.01 (Student's t test) comparing immunization with IL-12/ms vs. blank ms. b: Duration of IFNγ responses in CD4⁺ ILN cells 1-6 months after two immunizations with gonococcal OMV plus IL-12/ms or with OMV plus blank ms. Data shown as mean±SEM, N=3 samples, % of CD4⁺ cells staining for IFNγ; C, control ILN from unimmunized mice.

FIG. 9 shows resistance to gonococcal (FA1090) challenge persisted for at least 6 months after immunization with two doses of gonococcal (FA1090) OMV plus IL-12/ms. Left panel: recovery (CFU) of N. gonorrhoeae (mean±SEM, N=8 mice), *P<0.01 (ANOVA, gonococcal OMV plus IL-12/ms vs. gonococcal OMV plus blank ms); right panel: % of animals remaining infected at each time point, P<0.001 (Kaplan-Meier analysis, log-rank test, gonococcal OMV plus IL-12/ms vs. gonococcal OMV plus blank ms).

FIG. 10 shows resistance to heterologous gonococcal challenge. a: One month after immunization with FA1090 OMV plus IL-12/ms or blank ms, mice were challenged with N. gonorrhoeae strain FA1090 (homologous challenge) or strain MS11 (heterologous challenge). Left panel: recovery (CFU) of N. gonorrhoeae (mean±SEM, N=8 mice), *P<0.001 (ANOVA, for comparisons shown); right panel: % of animals remaining infected at each time point, P<0.02 for FA1090 challenge, IL-12/ms vs. blank ms; P<0.001 for MS11 challenge, IL-12/ms vs. blank ms (Kaplan-Meier analysis, log-rank test). b: Mice immunized with MS11 OMV were resistant to challenge with N. gonorrhoeae FA1090. Left panel: recovery (CFU) of N. gonorrhoeae (mean±SEM, N=8 mice), P<0.01 (ANOVA); right panel: % of animals remaining infected at each time point), P<0.01 (Kaplan-Meier analysis, log-rank test). c: Mice immunized with FA1090 OMV were resistant to challenge with N. gonorrhoeae FA19. Left panel: recovery (CFU) of N. gonorrhoeae (mean±SEM, N=8 mice), *P<0.01 (ANOVA, for comparisons shown); right panel: % of animals remaining infected at each time point, P<0.01, IL-12/ms vs. blank ms for FA1090 challenge; P<0.0001, IL-12/ms vs. blank ms for FA19 challenge (Kaplan-Meier analysis, log-rank test), N=8 mice. d: Mice immunized with FA19 OMV were resistant to challenge with N. gonorrhoeae FA1090. Left panel: recovery (CFU) of N. gonorrhoeae (mean±SEM, N=8 mice), P<0.01 (ANOVA); right panel: % of animals remaining infected at each time point), P<0.01 (Kaplan-Meier analysis, log-rank test). e: Mice immunized with FA1090 OMV were resistant to challenge with clinical isolate GC68. Left panel: recovery (CFU) of N. gonorrhoeae (mean±SEM, N=8 mice), P<0.01 (ANOVA); right panel: % of animals remaining infected at each time point), P<0.01 (Kaplan-Meier analysis, log-rank test).

FIG. 11 shows immunoproteomics of gonococal OMV. a.: SDS-PAGE of OMV preparations from N. gonorrhoeae strains FA1090, MS11, and FA19, stained with Coomassie blue. b.: Western blot analysis of mouse sera tested on gonococcal OMV preparations separated by SDS-PAGE. Lane 1, control serum from a mouse immunized with FA1090 OMV plus blank ms, tested against FA1090 OMV; lanes 2-4, serum #1 from a mouse immunized with FA1090 OMV plus IL-12/ms, tested against OMV from FA1090 (lane 2), MS11 (lane 3), or FA19 (lane 4); lane 5, serum #2 from a mouse immunized with FA1090 OMV plus IL-12/ms, tested against OMV from FA1090; lane 6, antibody H5 (anti-porin PIB3) tested against FA1090 OMV. c.-e.: proteome maps of gonococcal OMV derived from FA1090 (c), MS11 (d), and FA19 (e) revealed by 2D electrophoresis and Flamingo fluorescent staining (left panels) and their corresponding immunoblots (right panels) obtained by probing with mouse serum #2. Immunoreactive spots subjected to MS/MS analysis are labeled as spots 1 and 2 (arrows). Molecular mass marker (kDa) indicated on the left.

FIG. 12 shows resistance to challenge induced by immunization with gonococcal OMV plus IL-12/ms depended on IFNγ and B cells. a: Course of infection (FA1090) in IFNγ-ko vs wild-type mice immunized with FA1090 OMV; left panel, recovery (CFU) of N. gonorrhoeae (mean±SEM, N=8 mice), •P<0.01 (ANOVA); right panel, % of animals remaining infected at each time point, P<0.0001 for wild-type mice, IL-12/ms vs. blank ms (Kaplan-Meier analysis, log-rank test). b: Course of infection (FA1090) in μMT vs. wild-type mice immunized with FA1090 OMV; left panel, recovery (CFU) of N. gonorrhoeae (mean±SEM, N=8 mice), •P<0.01 (ANOVA); right panel, % of animals remaining infected at each time point, P<0.0001 for wild-type mice, IL-12/ms vs. blank ms (Kaplan-Meier analysis, log-rank test). Vaginal wash (c) and serum (d) antibody responses in IFNγ-ko vs wild-type (mean±SEM, N=5 samples) assayed at termination (day 13). IgA and IgG responses in vaginal wash and serum were significant (P<0.05, Student's t, OMV plus blank ms vs. OMV plus IL-12/ms) for wild-type mice, but not for IFNγ-ko mice. e: T cell cytokine responses in μMT vs. wild-type mice (mean±SEM, N=3 samples) assayed at termination (day 13). IFNγ response to immunization with OMV plus blank ms vs. OMV plus IL-12/ms was significant (P<0.01) for both wild-type and μMT mice (ANOVA). f: Course of infection (FA1090) in CD4-ko vs wild-type mice immunized with FA1090 OMV; left panel, recovery (CFU) of N. gonorrhoeae (mean±SEM, N=8 mice), #P<0.05, •P<0.01 (ANOVA) for comparisons shown; right panel, % of animals remaining infected at each time point, P<0.001 for wild-type mice IL-12/ms vs. blank ms, P<0.01 for CD4-ko mice IL-12/ms vs. blank ms (Kaplan-Meier analysis, log-rank test). g: Course of infection (FA1090) in CD8-ko vs wild-type mice immunized with FA1090 OMV; left panel, recovery (CFU) of N. gonorrhoeae (mean±SEM, N=8 mice), #P<0.05, •P<0.01 (ANOVA) for comparisons shown; right panel, % of animals remaining infected at each time point, P<0.001 for wild-type mice IL-12/ms vs. blank ms, P<0.02 for CD8-ko mice IL-12/ms vs. blank ms (Kaplan-Meier analysis, log-rank test).

FIG. 13 is a replicate of FIG. 1: I.vag. immunization with gonococcal OMV plus IL-12/ms induced resistance to genital infection with N. gonorrhoeae and generated an immune response. A: Mice were immunized 3 times at 7-day intervals with OMV (40 μg protein) from strain FA1090 plus control (blank) ms or IL-12/ms (1 μg IL-12); control mice were sham-immunized with either blank ms, or with IL-12/ms alone. Two weeks after the last immunization, all mice were challenged by i.vag. inoculation with N. gonorrhoeae strain FA1090 (5×10⁶ CFU), and infection was monitored by vaginal swabbing and plating. Left panel: recovery (CFU) of N. gonorrhoeae (mean±SEM, N=8 mice), P<0.01 (ANOVA, OMV plus IL-12/ms vs. OMV plus blank ms); right panel: % of animals remaining infected at each time point, P<0.001 (Kaplan-Meier analysis, log-rank test, OMV plus IL-12/ms vs. OMV plus blank ms). B: Vaginal wash (left) and serum (right) antibodies against strain FA1090 in samples collected after termination (day 15), shown as mean±SEM, N=5 samples. C: Intracellular cytokine staining in CD4⁺ cells recovered from ILN at termination (day 15), shown as mean±SEM, N=3 samples, % of CD4⁺ staining for each cytokine. D: Mice were immunized twice at a 14-day interval with gonococcal (Ngo) OMV (40 μg protein) plus blank ms or IL-12/ms (1 μg IL-12); control mice were sham-immunized with blank ms alone or with NTHI OMV (40 μg protein) plus IL-12/ms (1 μg IL-12). Two weeks later, all mice were challenged with N. gonorrhoeae FA1090. Left panel: recovery (CFU) of N. gonorrhoeae (mean±SEM, N=8 mice), *P<0.001 (ANOVA, gonococcal OMV plus IL-12/ms vs. gonococcal OMV plus blank ms); right panel: % of animals remaining infected at each time point, P<0.001 (Kaplan-Meier analysis, log-rank test, gonococcal OMV plus IL-12/ms vs. gonococcal OMV plus blank ms).

FIG. 14 shows examples of flow cytometry plots for data shown in FIG. 6C. Intracellular cytokine staining of ILN cells after clearance of infection (day 15) in mice immunized as shown. X-axes (FL4H): cytokine fluorescence; Y-axes (PE2): CD4 fluorescence (A-L); CD8 fluorescence (M-P).

FIG. 15 shows responses induced by immunization with OMV prepared from NTHI; samples collected 2 weeks after immunization as shown. A: Vaginal wash antibodies (mean±SEM, N=5 samples); B: serum antibodies (mean±SEM, N=5 samples); C: T cell cytokines in ILN cells (mean±SEM, N=3 samples). *P<0.01 vs. control samples (Student's t test).

FIG. 16 shows IgG subclass of anti-gonococcal antibody responses (mean±SEM, N=5 samples) in vaginal wash (A) and serum (B) induced by immunization with gonococcal OMV plus IL-12/ms. #P<0.05, *P<0.01 vs control samples (Student's t test).

FIG. 17 shows: A: Specificity of T cell responses in ILN for gonococcal antigen. ILN CD4+ T cells from mice immunized with FA1090 OMV plus blank ms or IL-12/ms were preloaded with CFSE and cultured in vitro in the presence of antigen-presenting cells for 3 days with (stim) or without FA1090 cells. Proliferation was assayed by flow cytometry after surface staining for CD4, and cytokine secretion was measured by ELISA. Data shown as mean±SEM, N=7 samples. *P<0.01 vs control samples (Student's t test). B: RT-PCR analysis of RNA extracted from vaginal tissue 3 days after the last immunization (1, 2, or 3 doses) with OMV plus blank ms or IL-12/ms. Data shown as mean±SEM, N=7 samples. *P<0.01 vs samples from mice immunized with OMV plus blank ms (Student's t test). C: Persistence of IFNγ response in CD4⁺ ILN cells harvested 1-6 months after immunization with OMV plus IL-12/ms. Data shown as mean±SEM, N=7 samples.

FIG. 18 shows responses in mice challenged with N. gonorrhoeae FA1090 6 months after immunization with FA1090 OMV plus IL-12/ms. A: Vaginal wash antibodies (mean±SEM, N=5 samples), B: serum antibodies (mean±SEM, N=5 samples), C: cytokine production by CD4⁺ ILN cells (mean±SEM, N=3 samples). #P<0.05, *P<0.01 vs control samples from sham-infected mice (Student's t test).

FIG. 19 shows responses in mice immunized with gonococcal OMV plus blank ms or IL-12/ms after heterologous gonococcal challenge. A, B, C: Mice immunized with FA1090 OMV and challenged with MS11; A: Vaginal wash antibodies, B: serum antibodies to MS11 (mean±SEM, N=5 samples); C: cytokine responses in CD4⁺ ILN cells (mean±SEM, N=3 samples). D, E, F: Mice immunized with FA1090 OMV and challenged with FA19; D: Vaginal wash antibodies, E: serum antibodies to FA19 (mean±SEM, N=5); F: cytokine responses in CD4⁺ ILN cells (mean±SEM, N=3 samples). #P<0.05, *P<0.01 vs control samples from sham-infected mice (Student's t test).

FIG. 20 is a replicate of FIG. 7A,B,F,G: I.vag. immunization with gonococcal OMV plus IL-12/ms in immunodeficient mice. A: Course of infection (FA1090) in IFNγ-ko vs wild-type mice immunized with FA1090 OMV; left panel, recovery (CFU) of N. gonorrhoeae (mean±SEM, N=8 mice), •P<0.01 (ANOVA); right panel, % of animal remaining infected at each time point, P<0.001 for wild-type mice, IL-12/ms vs. blank ms (Kaplan-Meier analysis, log-rank test). B: Course of infection (FA1090) in μMT mice immunized with FA1090 OMV; left panel, recovery (CFU) of N. gonorrhoeae (mean±SEM, N=8 mice). C: Course of infection (FA1090) in CD4-ko mice immunized with FA1090 OMV; left panel, recovery (CFU) of N. gonorrhoeae (mean±SEM, N=8 mice); right panel, % of animal remaining infected at each time point, P<0.01 (Kaplan-Meier analysis, log-rank test). D: Course of infection (FA1090) in CD8-ko vs wild-type mice immunized with FA1090 OMV; left panel, recovery (CFU) of N. gonorrhoeae (mean±SEM, N=8 mice); right panel, % of animal remaining infected at each time point, P<0.01 (Kaplan-Meier analysis, log-rank test).

FIG. 21 shows the effect of i.n. immunization with gonococcal OMV (strain FA19) plus IL-12/ms on gonococcal challenge infection in BALB/c mice. A: recovery (CFUs) of N. gonorrhoeae (mean±s.e.m., N=8 mice), P<0.01 (ANOVA, gonococcal OMV plus IL-12/ms vs. gonococcal OMV plus blank ms); B: percentage of animals remaining infected at each time point.

FIG. 22 shows the effect of immunization with different doses of gonococcal OMV (strain FA1090) plus IL-12/ms on heterologous gonococcal challenge infection with strain FA19 in BALB/c mice. Low dose: 60 μg, mid dose: 30 μg, low dose: 15 μg of OMV protein per dose, plus 1 μg IL-12/ms. Left panel: recovery (CFUs) of N. gonorrhoeae (means±s.e.m., N=8 mice); right panel: percentage of animals remaining infected at each time point.

FIG. 23 shows A: comparison of the effects of intranasal or intravaginal immunization with gonococcal OMV (30 μg protein; strain FA1090) plus 1 μg IL-12/ms on heterologous gonococcal challenge infection with strain FA19 in BALB/c mice. Left panel: recovery (CFUs) of N. gonorrhoeae (means±s.e.m., N=8-9 mice); right panel: percentage of animals remaining infected at each time point. B: serum, C: vaginal wash, D: salivary antibodies against N. gonorrhoeae in samples collected after clearance of the infection at termination (2 weeks after challenge); *P<0.01 compared with controls (Student t test).

FIG. 24 shows a comparison of the effects of intranasal or intravaginal immunization with gonococcal OMV (30 μg protein; strain FA1090) plus 1 μg IL-12/ms on homologous gonococcal challenge infection with strain FA1090 in BALB/c mice. Left panel: recovery (CFUs) of N. gonorrhoeae (means±s.e.m., N=8-9 mice); right panel: percentage of animals remaining infected at each time point.

FIG. 25 shows the effect of intranasal immunization with gonococcal OMV (30 μg protein, strain FA1090) plus 1 μg IL-12/ms on heterologous gonococcal challenge infection with strain MS11 in BALB/c mice. Mice were given either one dose of OMV plus IL-12/ms, or two doses with a 2 week interval; control mice were given 2 doses of blank ms. Left panel: recovery (CFUs) of N. gonorrhoeae (means±s.e.m., N=9 mice); right panel: percentage of animals remaining infected at each time point.

FIG. 26 shows the effect of intranasal immunization with gonococcal OMV (30 protein, strain MS11) plus 1 μg IL-12/ms on heterologous gonococcal challenge infection with strain FA1090 in BALB/c mice. Left panel: recovery (CFUs) of N. gonorrhoeae (means±s.e.m., N=8-9 mice); right panel: percentage of animals remaining infected at each time point.

FIG. 27 shows the effect of intranasal immunization with gonococcal OMV (30 μg protein, strain MS11) plus 1 μg IL-12/ms on heterologous gonococcal challenge infection with strain FA19 in BALB/c mice. Left panel: recovery (CFUs) of N. gonorrhoeae (means±s.e.m., N=9 mice); right panel: percentage of animals remaining infected at each time point.

### DESCRIPTION OF THE INVENTION

The present invention is based on our studies which have helped to unfold the ways in which N. gonorrhoeae prevents the immune system from mounting effective immune responses against it. We provide here a novel approach to overcome the ability of N. gonorrhoeae to suppress immune response against it.

In an aspect, the method comprise reducing the likelihood of an individual contracting a N. gonorrhoeae infection by administration of an antigenic preparation from N. gonorrhoeae and IL-12 present in microspheres (ms). An example of an antigenic preparation from N. gonorrhoeae is OMVs. The OMVs and the IL-12 ms may be delivered intravaginally. In one embodiment, the OMVs and the IL-12 ms may be delivered intranasally. The OMVs and the IL-12 ms may be delivered by separate routes of administration. For example, one may be delivered intravaginally and the other intranasally. In one embodiment, the OMVs and the IL-12 ms are delivered by the same route of administration, e.g, intranasally.

In an embodiment, the OMVs and the IL-12 ms are delivered in the same composition. Amounts of OMVs and IL-12 ms per dose can be such that an immune response is elicited. The OMVs can be in the range of 0.01 to 1 mg of protein per dose. In one embodiment, the OMVs can be in the range of 0.01 to 2 mg of protein per dose. In embodiments, OMVs can be 10, 50, 100, 250, 500, 750, 1,000, 1250, 1,500, 1,750 or 2,000 μg protein per dose. In an embodiment, OMVs can be in the range of 15-300 μg protein per dose. In one embodiment, the OMV dose can be 50 to 1,000 μg protein. For a 70 kg human, that corresponds to a OMV dose of about 0.7 to 28 microgram protein per kg body weight. In one embodiment, the OMVs can be from 0.5 to 30 μg protein/kg body weight. The IL-12 (in microspheres) can be 1 to 500 μg/dose. That corresponds to about 14 ng to 7 μg/kg body weight (assuming a 70 kg body weight). In an embodiment, IL-12 can be 10 ng to 10 μg/kg body weight. In one embodiment, the IL-12 can be 10, 50, 100, 200, 300, 400 or 500 μg/dose. In an embodiment, the IL-12 can be 10 to 300 ng or 5 to 300 ng per kg body weight. In an embodiment, IL-12 can be 0.5 to 20 micrograms in microspheres per dose. Determining the amount of microspheres can be done based on the loading of IL-12. The compositions may be provided in carriers, buffers and the like or may be lyophilized. The two components may be provided separately, and can be combined just before administration or may be administered separately. In one embodiment, the composition does not have any added free soluble IL-12. Any leaked IL-12 from the microspheres prior to administration is considered to be insignificant. Even if free soluble IL-12 is present, it is not considered to contribute to the present effects and method. Rather, encapsulated IL-12 in microspheres was required in the composition to see the protective effects.

Outer membrane vesicles can be prepared from the outer membranes of N. gonorrhoeae. For example, OMVs can be prepared from the outer membranes of a cultured strain of Neisseria gonorrhoeae spp. OMVs can be obtained from a N. gonorrhoeae grown in broth or solid medium culture. The preparation may comprise separating the bacterial cells from the culture medium (e.g. by filtration or by a low-speed centrifugation and the like), lysing the cells (e.g. by addition of detergent, osmotic shock, sonication, cavitation, homogenization and the like) and separating an outer membrane fraction from cytoplasmic molecules (e.g. by filtration; or by differential precipitation or aggregation of outer membranes and/or outer membrane vesicles, or by affinity separation methods; or by a high-speed centrifugation).

The compositions can be administered preferably as multiple doses with an interval in between. For example, at least two doses can be administered with an interval of at least one week in between. The interval may be from one to three weeks. In one embodiment, two doses are used with an interval of about 2 weeks in between.

In one embodiment, the IL-12 formulations of the present invention are sustained release formulations. In one embodiment, IL-12 is delivered as incorporated (also referred to herein as encapsulated or microencapsulated) in polymeric microparticles (also referred to herein as microspheres). In one embodiment, the microparticles are biodegradable and biocompatible. Preparation techniques for such microspheres are known in the art. See for example, U.S. Pat. Nos. 6,143,211; 6,235,244; 6,616,869; and 7,029,700, the disclosures of which pertaining to methods and compositions for preparation of microspheres are incorporated herein by reference. In one embodiment, a phase inversion technique is used to prepare microencapsulated IL-12. In general, a biodegradable polymer is dissolved in a solvent (such as dichloromethane or other organic solvent) and then a mixture is formed by adding micronized IL-12 (i.e. lyophilized mixtures of IL-12 and excipient such as polyvinyl pyrrolidone) to the polymer dissolved in the solvent. A non-solvent (such as alcohol or hexane) is then introduced causing spontaneous formation of microencapsulated IL-12. Examples of biodegradable polymers include polymers of lactic acid and glycolic acid, polyanhydrides, poly(ortho)esters, polyurethanes, poly(butyric acid), poly(valeric acid), poly(caprolactone), poly(hydroxybutyrate), poly(lactide-co-glycolide) and poly(lactide-co-caprolactone), and natural polymers. In one embodiment, the microspheres are composed of a polymer of lactic acid (polylactic acid (PLA)).

In one embodiment, the IL-12 containing microspheres degrade by hydrolysis slowly over time, releasing the encapsulated IL-12. The microspheres are suspended before use and can also be delivered in an acceptable buffered physiological saline solution. In one embodiment, slow release of IL-12 over a period of time such as approximately 4 days allows for continuous stimulation of locally present immune cells without elevating the concentration of IL-12 in the local tissues or the circulation to potentially harmful levels. The microspheres are made of biodegradable materials. In one embodiment, the hydrolytic product of the microspheres is lactic acid, a harmless product of normal metabolism. PLA is a component of absorbable sutures and has been in use for that purpose for many decades, and is therefore considered safe. Microencapsulated IL-12 has been shown to be stable in storage at ambient temperatures and to have a long shelf-life.

The microspheres are in the range of 10 nm to 10 microns. The microspheres may be suspended in pharmaceutically acceptable medium such as a physiological buffer. In one embodiment, the loading of IL-12 is from 0.1 to 10 μg per mg of the particles. In one embodiment, the loading is from 1 to 5 μg IL-12 per mg of the particles. In one embodiment, the loading is from about 2.5 μg IL-12 per mg of the particles.

The IL-12 formulations can be used in amounts that will result in therapeutic and/or prophylactic effects. An effective dose in mice was observed to be 1 μg of IL-12. Determining the effective dosage for humans is within the purview of clinicians and other individuals involved in the treatment of such infections. Generally, the amount administered depends upon various factors including the severity of the infection, the weight, health and age of the individual. Such factors can be readily determined by a clinician. In one embodiment, the dose may be from 1 μg to 200 μg of IL-12 per day. In some embodiments, the dose is 1, 5, 10, 15, 20, 50, 75, 100, 125, 150, 175 and 200 μg of IL-12 per dose and all integers between 1 and 200 μg and all ranges therebetween. The dosage required may be less if used in conjunction with an antimicrobial agent.

The dosage may be repeated as necessary. For example, the administration may be repeated daily, multiple times in a day, or at longer intervals, such as at intervals of 2-4 days, weekly or monthly. In one embodiment, the administration is repeated at intervals from 1 day to 1 month (28, 29, 30 or 31 days) or beyond that and all intervals therebetween. The treatment regimen may be repeated as necessary. In some embodiments, the dosage is administered every 2, 3, 4, 5, 6, 7, 10, or 14 days, or longer.

In one embodiment, the present invention provides a method of treating genital tract infections in a female subject by intravaginal application of IL-12 incorporated in polymeric microspheres. The infections that can be treated by this method include bacterial, fungal, parasitic, viral and the like. In one embodiment, the amount is sufficient to promote Th1-driven response against the microorganisms causing the infection. In one embodiment, the amount is sufficient to provide a therapeutic effect, a prophylactic effect, or both against the causative microorganisms. The term “treated” or “treatment” as used herein means to reduce or eliminate an infection. An infection is considered to be reduced when the underlying cause of the infection is reduced.

In one embodiment, the method of the present invention is useful for treating genital tract infections, such as cervico-vaginal infections, caused by bacteria, such as N. gonorrhoeae. The method comprises the steps of providing local (intravaginal) application of the cytokine interleukin-12 (IL-12) incorporated in biodegradable, biocompatible microspheres. In one embodiment, the dose is sufficient to promote Th1-driven immune responses against infection with N. gonorrhoeae. In one embodiment, the invention provides a method for therapy or prophylaxis or both for cervico-vaginal gonococcal infection (i.e., gonorrhea) by means of local administration of IL-12 microspheres. While not intending to be bound by any particular theory, it is considered that this method works, at least in part, by reversing the ability of N. gonorrhoeae to interfere with the host's immune responses.

In one embodiment, the IL-12 formulation is delivered locally to the mucosal surface of the genital tract of an individual. In one embodiment, the individual is not already receiving IL-12, or has not been administered IL-12 prior to the initiation of the present method. In one embodiment, the individual is not receiving IL-12 via any other administration mode. In one embodiment, the formulation contains no other therapeutic agent, no other prophylactic agent, or no other agent that is both therapeutic and prophylactic. In one embodiment, the formulation does not contain the infection causing microorganism (such as in an inactivated form) or an antigen therefrom, and the individual has not been and/or is not being administered the inactivated microorganism or an antigen therefrom. In another embodiment, the formulation may be delivered to an individual who is already receiving treatment (other than IL-12) for genital tract infection (such as gonococcal infection).

In one embodiment, the invention further comprises the step of administering an antimicrobial agent to the individual. For example, in one embodiment, the method of this invention comprises the steps of identifying an individual who is suffering from or has been diagnosed with an infection of the genital tract, delivering to the genital tract locally (such as intravaginally) a composition comprising a therapeutically effective, a prophylactically effective, or both therapeutically and prophylactically effective amount of a composition comprising IL-12 in biodegradable polymeric microspheres, and optionally administering to the individual one or more antimicrobial agents (such as antibiotics, antifungal or antiviral agents). The antimicrobial agents may be administered prior to, during or after the administration of the IL-12 formulation. An example of such a treatment is the administration of antibacterial agents, such as antibiotics. Examples of suitable antibiotics used for genital tract infections include fluorquinolones, cephalosporins, azithromycin, Ceftriaxone, doxycycline, and Cefixime.

In one embodiment, the administration of the microencapsulated IL-12 as described here reduces the N. gonorrhoeae infection. In one embodiment, the infection is eliminated. The presence or absence of infection or the level of infection may be tested by routine microbiological methods (such as culture and testing). In one embodiment, the infection may be tested by obtaining vaginal swab and testing for the presence of bacteria (such as by the ability to form colonies), or by nucleic acid amplification methods.

In another embodiment, the administration of the microencapsulated IL-12 as described here reduces the N. gonorrhoeae infection and reduces the risk of repeat infection of N. gonorrhoeae after the treatment with microencapsulated IL-12 has been stopped. While not intending to be bound by any particular theory, it is considered that the prophylactic effect of IL-12 is achieved by stimulation of the immune system. In one embodiment, the administration of IL-12 does not significantly increase the level of IL-12 in the systemic circulation. In one embodiment, the serum level of IL-12 does not increase to greater than 50 picograms/ml.

For intravaginal applications, the formulations of the present invention can be delivered as applied to an article of manufacture acting as a carrier. For example, the formulations may be incorporated into or onto and then delivered via an insert, an applicator, tablet, suppository, vaginal ring, vaginal sponge, tampon and the like. The formulation may also be delivered in the form of a liquid, cream, gel, lotion, ointment, paste, spray and the like.

The pharmaceutical formulations may optionally include pharmaceutically acceptable carriers, buffers, diluents, solubilizing or emulsifying agents, and various salts. Such additives are well known in the art. See, e.g., Remington's Pharmaceutical Sciences, 18th Ed. (1990, Mack Publishing Co., Easton, Pa. 18042).

An advantage of local application of microencapsulated IL-12 as described herein is that it can provide a sustained effect while avoiding problems of potential systemic toxicity.

In one embodiment, the present invention is used for treating genital Chlamydia trachomatis infection (chlamydia). Chlamydia is another sexually transmitted disease (STD) of even more frequent occurrence than gonorrhea, and is the most frequently reported infectious disease in the US, thought to affect up to 3 million individuals per annum (>92 million worldwide). It is also a major cause of pelvic inflammatory disease in women and its sequelae (infertility and risk for ectopic pregnancy). Therefore in one embodiment, local (intravaginal) application of IL-12 incorporated in polymeric microspheres is used to promote local Th1-immune responses for therapy and prophylaxis against C. trachomatis. In one embodiment, the method of the present invention is used to treat urinogenital infections due to N. gonorrhoeae and C. trachomatis. This may be advantageous in the STD clinic setting because gonorrhea and chlamydia present with similar signs and symptoms, and the differential diagnosis may depend on identifying the causative organism. Furthermore, mixed infections with both are common. In other embodiments, other genital tract infections could also be treated with (intravaginal) application of microencapsulated IL-12 to enhance local immunity against them.

In other embodiments, local application of microencapsulated IL-12 is used in the treatment of other local mucosal infections where the normal immune response is insufficient to eliminate them. Examples include: bronchitis and chronic obstructive pulmonary disease (respiratory tract), otitis media (middle ear infection, which is the most frequent reason for pediatric office visits in the US), Helicobacter pylori infection (which causes gastric ulcer and can lead to gastric cancer), and possibly periodontal disease (which afflicts most adults from age 35 onwards and is the main cause of tooth loss in adults).

In one embodiment, the IL-12 may be administered with a gonococcal antigen containing vaccine. For example, IL-12 can be administered with a locally administered non-living gonococcal vaccine. As described in Example 3, we have immunized mice i.vag. with a gonococcal outer membrane vesicle (OMV) preparation administered with or without IL-12/ms. OMV contain most of the surface antigens in native conformation, not denatured by heat or chemical inactivation. The results demonstrate the generation of a Th1-driven, antibody-dependent, protective immune response that persists for at least several months and is effective against antigenically diverse strains of N. gonorrhoeae. As described in Example 4, immunization can also be carried out via intranasal route.

The OMV and the IL-12 microspheres may be administered to female or male subjects. When administered to a male subject, the compositions may be delivered intranasally, and when administered to a female subject, the compositions may be delivered intravaginally and/or intranasally.

The following examples are provided to further illustrate the disclosure. These examples are not intended to be restrictive.

### Example 1

The invention has been demonstrated in the mouse model of vaginal gonococcal infection. Details of the mouse model can be found in Jerse, Infect. Immun. 67: 5699-5708; 1999.

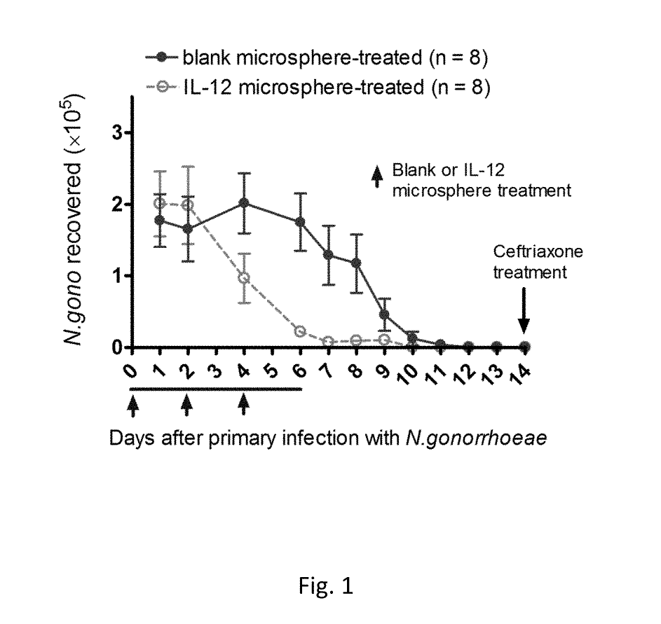

Intravaginal treatment of mice with IL-12 microspheres (1 μg) on days 0, 2, and 4 of primary vaginal infection (on day 0) with N. gonorrhoeae resulted in accelerated clearance of the infection, compared to control mice given blank microspheres (See FIG. 1).

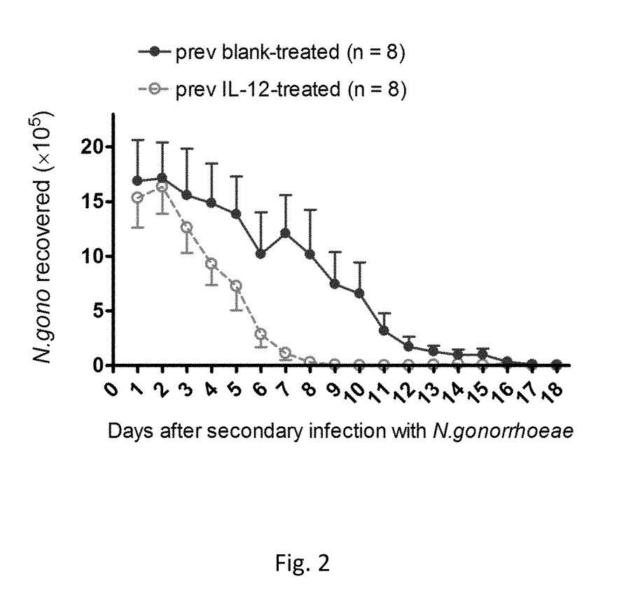

FIG. 1 illustrates the effect of intravaginal treatment with 1 μg of IL-12 encapsulated in PLA microspheres (on days 0, 2, and 4) on the course of vaginal infection with N. gonorrhoeae in mice. Data shown as mean±SEM cfu of N. gonorrhoeae recovered from vaginal swabs taken daily; N=8 mice per group. Control mice were given blank microspheres. Mice were treated with antibiotic on day 14 and then rested for secondary infection (See FIG. 2).

When mice that were treated with IL-12 microspheres during primary vaginal gonococcal infection were allowed to recover, treated with antibiotic (ceftriaxone) on day 14, rested and then reinfected one month later with N. gonorrhoeae, the secondary infection was cleared faster than in control mice given blank microspheres during primary infection (See FIG. 2). Normally, secondary infection is considered to clear with the same kinetics as primary infection, and there is little or no antibody response developed.

FIG. 2 illustrates the effect of intravaginal treatment with IL-12 microspheres during primary infection with N. gonorrhoeae (See FIG. 1) on the course of secondary vaginal infection with N. gonorrhoeae in mice. Control mice were given blank microspheres. Data shown as mean±SEM cfu of N. gonorrhoeae recovered from vaginal swabs taken daily; N=8 mice per group.

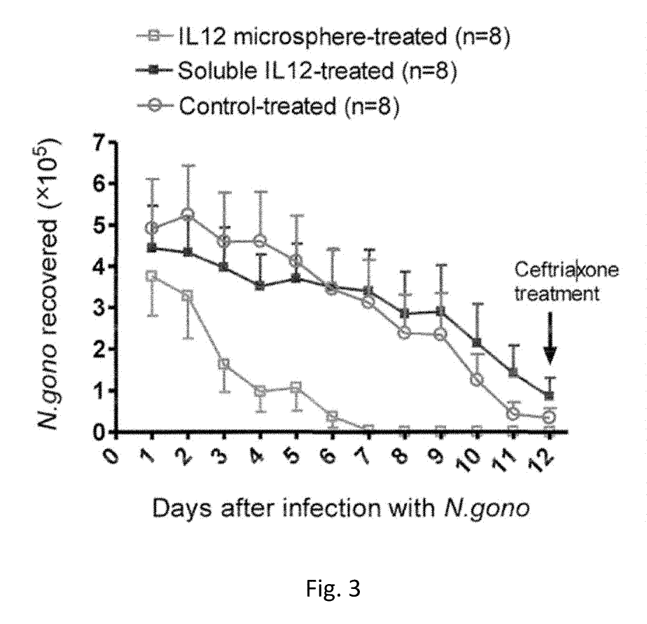

Further, the effect of intravaginal treatment with microencapsulated IL-12 (IL-12 microspheres) was compared with soluble IL-12 on the course of mouse genital tract infection with Neisseria gonorrhoeae. For this purpose, 1 μg of IL-12 was instilled intravaginally in a group of 8 mice in free soluble form (dissolved in sterile phosphate-buffered physiological saline) on days 0, 2, 4, 6, 8, and 10 after infection with N. gonorrhoeae (i.e., every other day until the infection was cleared), in direct comparison with mice treated with IL-12 microspheres and a control group treated with vehicle only. Mice treated with IL-12 microspheres cleared the infection within 7 days, much faster than the control group, whereas mice treated with soluble IL-12 cleared the infection at the same rate as the control group (See FIG. 3). Data shown as mean±SEM cfu of N. gonorrhoeae recovered from vaginal swabs taken daily; N=8 mice per group.

The results show that local intravaginal treatment with soluble IL-12 had no effect on the course of infection, whereas IL-12 microspheres accelerated clearance, as described previously.

### Example 2

This example describes another set of experiments that illustrate the effectiveness of intravaginal application of IL-12 microspheres on N. gonorrhoeae vaginal infection.

### Materials and Methods

Mice:

BALB/c mice were purchased from Jackson Laboratories (Bar Harbor, Me.), and were maintained under standard conditions in the Laboratory Animal Facility at the University at Buffalo. All animal use protocols were approved by the Institutional Animal Care and Use Committee of the University at Buffalo.

Bacteria:

N. gonorrhoeae FA1090 were cultured on GC agar supplemented with hemoglobin and ISOVITALEX®, an enrichment medium (BD Diagnostic Systems, Franklin Lakes, N.J.). Growth was checked for colony morphology consistent with Opa protein and pilus expression, and gonococci were harvested from plates and the cell density was determined. Opa expression as was: Opa A, B/D/G, E/K.

Microspheres:

Cytokines were encapsulated into poly-lactic acid (PLA) microspheres using the Phase Inversion Nanoencapsulation (PIN) technology as follows. Briefly, recombinant IL-12 (mouse or human) is mixed with excipients including sucrose (0.1%, w/w) and polyvinylpyrrolidone in water and then is lyophilized. The lyophilized material is dissolved in tertyl butyl alcohol (TBA) and is mixed with polylactic acid (PLA) resomer dissolved in TBA (1 to 3 ratio, vol:vol for micronized IL-12 and PLA solution). This solution is then poured into 100× volume of heptane to induce formation of the particles. The particles are then filtered and lyophilized. Three formulations were produced: (a) control microspheres containing no cytokine or antibody; (b) murine IL-12 (0.25 μg/mg particles); and (c) murine IL-17 (0.25 μg/mg particles).

Mouse Vaginal Infection Model:

Female mice between 7 and 9 weeks old were infected vaginally on day 0 with live N. gonorrhoeae FA1090 as previously described. Vaginal mucus was quantitatively cultured daily on GC agar supplemented with selective antibiotics to determine the bacterial colonization loads. The limit of detection was 100 CFU recovered per mouse. Intravaginal treatments with microsphere preparations were given every second day from day 0 to day 8, by instillation of 40 μl suspensions in PBS of microspheres containing IL-12 or IL-17, or control microspheres.

Cell Isolation and Flow Cytometry:

Mice were sacrificed and the iliac lymph nodes (ILN) and genital tracts were excised aseptically. ILN were teased in Hanks' buffered salt solution to release cells. Vaginal single-cell suspensions were prepared by enzymatic digestion. Isolated cells were washed with staining buffer twice, then incubated with the indicated antibodies for 30 min on ice, washed twice, and analyzed on a FACSCalibur cytometer. For determination of intracellular cytokine expression, cells were restimulated with phorbol myristate acetate-ionomycin-GOLGISTOP®, a protein transport inhibitor (eBioscience, San Diego, Calif.) for 5 h, and then fixed with CYTOFIX/CYTOPERM®, a fixation/permeabilization solution, (eBioscience). Antibodies to mouse CD4, CD8, CD19, CD11b, CD11c, NKG2D, Gr-1, IFN-γ, IL-4, and IL-17A conjugated with fluorescein isothiocyanate, phycoerythrin, or allophycocyanin were purchased from eBioscience.

Cytokine ELISA:

IL-12p70, IFN-γ, IL-4, IL-5, and IL-17A levels in serum or vaginal wash samples were measured in triplicate using ELISA kits purchased from eBioscience.

Real-Time RT-PCR:

Total cellular RNA of whole vaginas harvested from the mice was isolated with RNEASY®, RNA purification Mini Kits (Qiagen, Valencia, Calif.), and was transcribed to cDNA using the ISCRIPT™ cDNA synthesis kit (Bio-Rad, Hercules, Calif.). Real-time RT-PCR was performed on an ICYCLER IQ® real-time PCR detection system (Bio-Rad) using SYBR® Green dye, a fluorescent dye, (Bio-Rad) for real-time monitoring of the PCR. Relative quantification of target genes was analyzed based on the threshold cycle (Ct) determined by Bio-Rad IQ™ 5 optical system software.

Assay of Serum and Mucosal Antibodies:

Samples of saliva, vaginal wash, and serum were collected from individual mice on day 15 post-inoculation. Gonococcus-specific IgA, IgG, and IgM in saliva, sera, and vaginal washes and total IgA, IgG, and IgM concentrations in secretions were assayed by ELISA.

Statistical Analysis:

Data are expressed as the means±standard errors of the means (SEM). Data on the effects of IL-12-, IL-17-, anti-TGF-β-, anti-IL-10-loaded versus blank microsphere treatments on vaginal N. gonorrhoeae infection were analyzed using repeated-measures analysis of variance (ANOVA) with Bonferroni corrected post-hoc testing of pair-wise comparisons. Kaplan-Meier analysis with log-rank testing was also used to compare infection clearance. Data from in vitro experiments were analyzed by unpaired two-tailed t tests to compare the mean values between two selected groups. P<0.05 was considered statistically significant.

### Results

Intravaginal Administration of IL-12 Microspheres Protects Mice Against Genital Tract N. gonorrhoeae Infection.

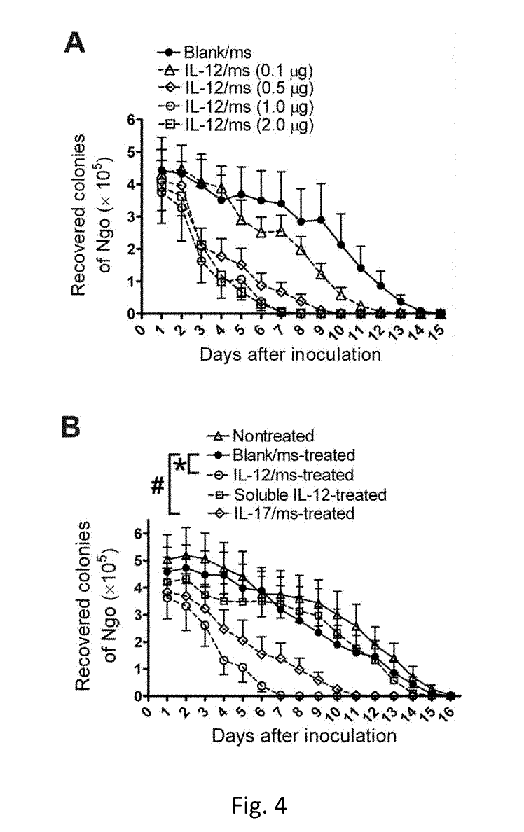

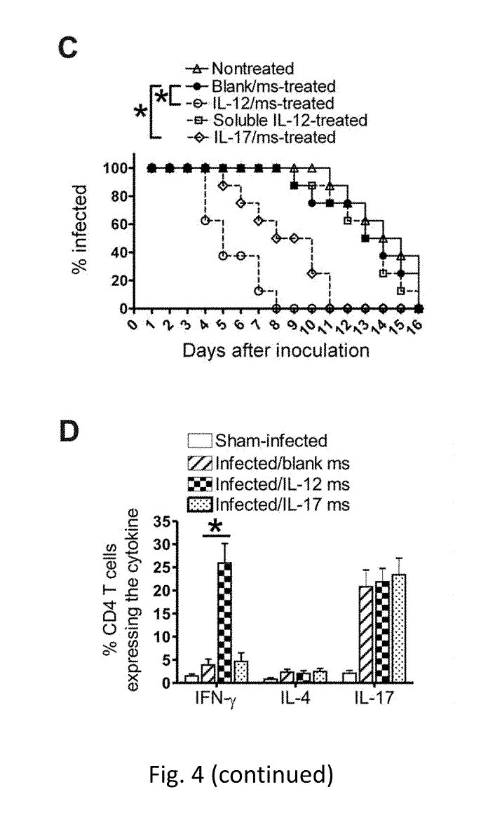

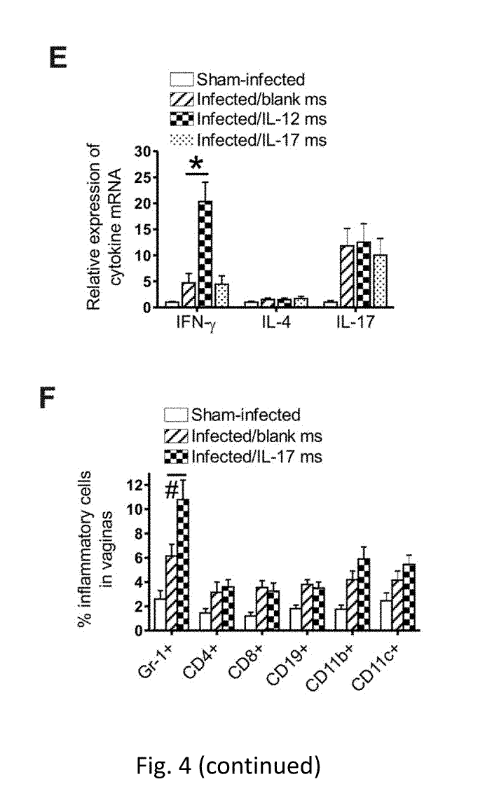

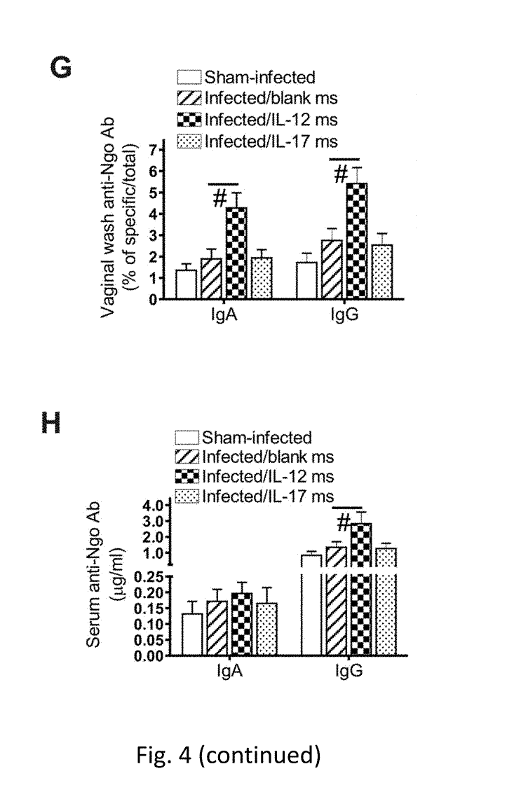

To examine the therapeutic effect of IL-12-loaded microspheres, groups of female BALB/c mice were infected with N. gonorrhoeae and the bacterial burden was monitored daily by vaginal swab culture. Preliminary dose-ranging experiments showed that intravaginal instillation of microspheres containing 1.0 μg of IL-12 every second day was sufficient to accelerate clearance of the infection relative to treatment with blank microspheres; no further enhancement of clearance was obtained with 2.0 μg of microencapsulated IL-12, and lower doses were progressively less effective (FIG. 4A).

Untreated or blank microsphere-treated mice cleared the infection in ˜15 days (FIG. 4B, C). Intravaginal instillation of microencapsulated IL-12 at the optimal 1.0 μg dose significantly reduced the recoverable N. gonorrhoeae load starting from day 4, and these mice cleared the infection by day 7, 8 days earlier than blank microsphere-treated or untreated mice (FIG. 4B, C). The infection did not relapse after treatment ceased on day 7. In contrast, intravaginal administration of free, soluble IL-12 was completely ineffective in enhancing clearance of N. gonorrhoeae (FIG. 4B, C).

Intravaginal administration of IL-17-loaded microspheres at the optimal dose (1.0 μg) also accelerated clearance of N. gonorrhoeae infection, but to a lesser extent than IL-12 microspheres given on the same schedule (FIG. 4B, C).

Treatment with IL-12 Microspheres Enhances Th1 and Antibody Responses to Vaginal Gonococcal Infection.

To elucidate the mechanisms underlying the therapeutic effects of IL-12, we characterized the local immune responses to genital gonococcal infection in mice treated with IL-12-loaded or blank microspheres. Single-cell suspensions were prepared from ILN and vaginas of 7 mice per group at 3, 5, 7, and 14 days after inoculation with N. gonorrhoeae or vehicle only for evaluation by flow cytometry to detect intracellular IFN-γ, IL-4, and IL-17. Starting on day 3 after inoculation, IL-17⁺/CD4⁺ T cells were observed in the local draining ILN, with production peaking at day 5 and continuing for the duration of infection. At day 5, approximately 22% of CD4⁺ T cells present in the ILN of control-treated infected mice were IL-17⁺, whereas only ˜3.5% were IFN-γ⁺ and few IL-4⁺/CD4⁺ T cells were detected (FIG. 4D). IL-12 microsphere treatment markedly enhanced Th1 immune responses to N. gonorrhoeae, indicated by significantly increased numbers of IFN-γ⁺/CD4⁺ T cells (FIG. 4D). In contrast, IL-12 microspheres did not change Th2 or Th17 responses as the numbers of IL-4⁺/CD4⁺ and IL-17⁺/CD4⁺ T cells in ILN were similar between the treated groups (FIG. 4D). RT-PCR analyses showed that IFN-γ, but not IL-4 or IL-17 mRNA expression was elevated in the vaginas of infected mice following IL-12 microsphere treatment (FIG. 4E). Although IL-17 microspheres ameliorated gonococcal infection, this treatment was not associated with enhanced Th1 or Th2 responses (FIG. 4D, E), but there was increased influx of Gr-1⁺ neutrophils into the genital tract (FIG. 4F).

We also measured by ELISA IL-12p70, IFN-γ, IL-4, and IL-17 concentrations in vaginal wash and serum collected 7 days after inoculation. IL-12 (176.5±48.6 pg/ml) was detected in vaginal wash from infected mice treated with IL-12 microspheres. Low levels of IL-12 (41.7±10.7 pg/ml) were found in the serum of these mice, suggesting that the effects of IL-12 microsphere treatment on gonococcal infection did not result primarily from the passage of the cytokine into the circulation. Consistent with the flow cytometric studies, IFN-γ was present in the vaginal wash (32.6±9.8 pg/ml) and serum (43.3±11.5 pg/ml) of infected mice treated with IL-12 microspheres, but IL-4 and IL-17 were not detected. None of these cytokines was detected in control-treated infected mice.

IL-12 can stimulate humoral immune responses in an IFN-γ-dependent manner or directly. We therefore determined whether IL-12 microsphere treatment during N. gonorrhoeae infection led to the production of anti-gonococcal antibodies in vaginal wash, saliva, and serum collected 15 days after inoculation. IgM antibodies were at low levels with little difference between experimental groups (data not shown). No salivary gonococcus-specific antibody was detected in any group of mice (data not shown). N. gonorrhoeae infection of control-treated mice did not significantly elevate gonococcus-specific IgA or IgG antibodies in either vaginal washes or sera. However, IL-12 microsphere treatment increased vaginal and serum specific IgG antibody (FIG. 4G, H), as well as vaginal specific IgA antibody production (FIG. 4G).

Treatment with IL-12 Microspheres Induces Protective Anamnestic Immunity Against Secondary N. gonorrhoeae Infection.

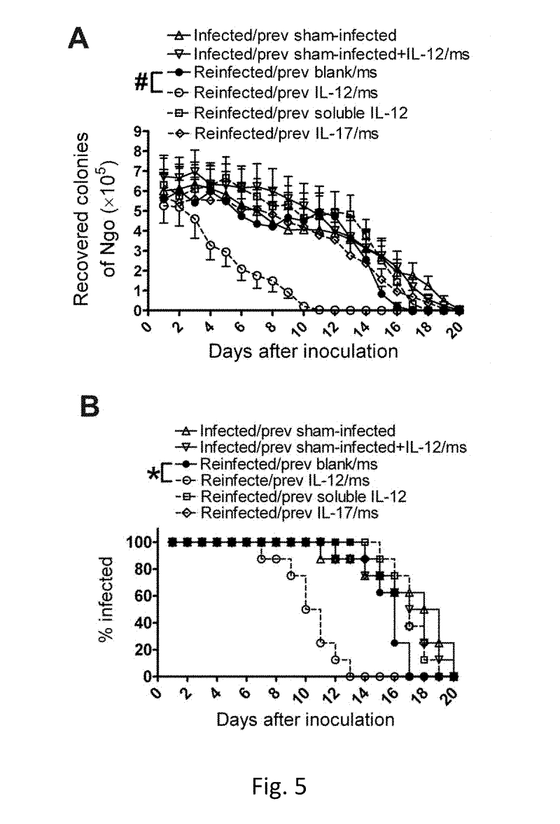

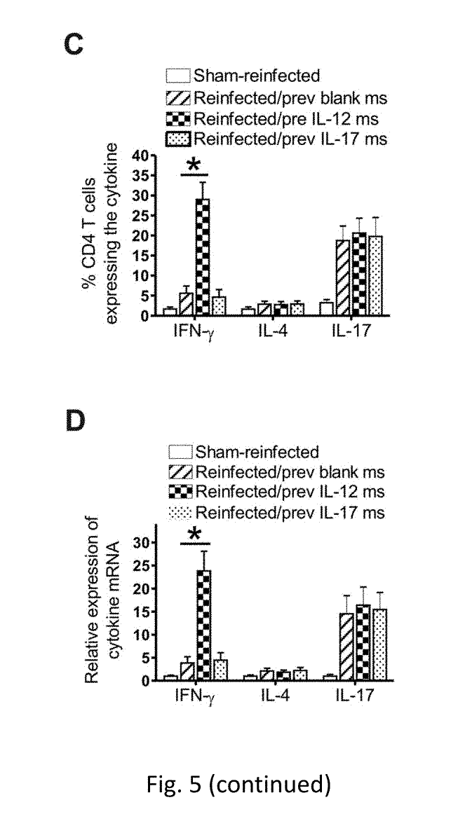

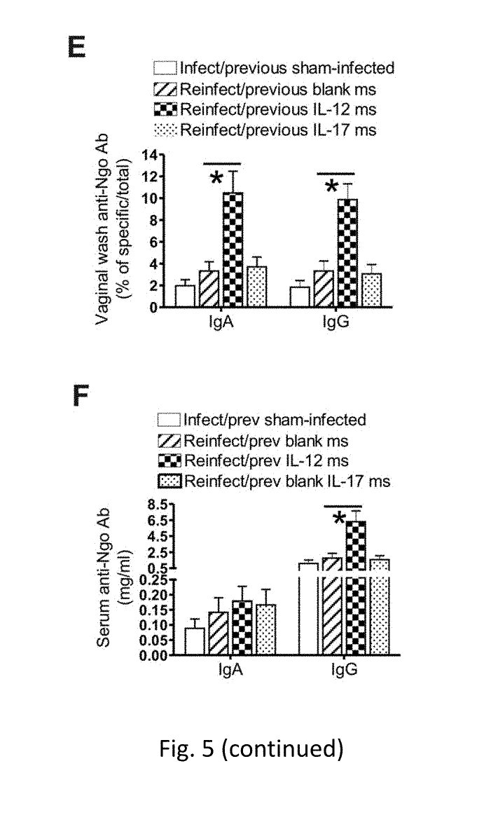

We further assessed whether IL-12 microsphere treatment resulted in the generation of immune memory and protective immunity against reinfection. Groups of mice infected with N. gonorrhoeae were treated with IL-12-loaded or blank microspheres, and after the infection had run its course, the mice were treated with ceftriaxone (300 μg i.p.) on day 15 to ensure complete elimination of the gonococci. An additional group of sham-infected mice treated with IL-12 microspheres was used to evaluate the possible persistent effect of IL-12 in the absence of infection. Five to six weeks later, all mice were inoculated with N. gonorrhoeae of the same strain without any further treatment. As observed previously, primary infection of control-treated mice did not protect them against subsequent secondary challenge: the duration and bacterial burden of secondary gonococcal infection in previously blank microsphere-treated mice were the same as for the primary infection of age-matched naïve mice (FIG. 5A, B). In contrast, intravaginal treatment with IL-12-loaded microspheres during primary infection protected the mice against secondary infection: reinfected mice that had been treated with IL-12 microspheres during the primary infection resisted the challenge more effectively than controls (FIG. 5A, B). However, previous IL-12 microsphere treatment of sham-infected mice did not induce protection against subsequent infection (FIG. 5A, B). This result also excluded the possibility that any persisting microspheres still affected the secondary N. gonorrhoeae infection.

Flow cytometric and RT-PCR analyses of ILN cells and vaginas taken on day 5 and day 3 of secondary infection, respectively, indicated that the protective effect of previous IL-12 microsphere treatment on secondary gonococcal infection was also associated with significantly enhanced Th1 (IFN-γ) responses (FIG. 5C, D). There was also a robust specific secondary antibody response in IL-12 microsphere-treated mice after they were rechallenged with N. gonorrhoeae. Gonococcus-specific IgA and IgG antibodies in vaginal washes and IgG antibodies in sera of reinfected mice previously treated with IL-12 microspheres were significantly higher than those of control groups (FIG. 5E, F).

In contrast to the effects of IL-12 microsphere treatment, treatment with IL-17 microspheres during primary gonococcal infection did not lead to any protective immunity to secondary gonococcal infection, or induce any anamnestic T cell or antibody responses (FIG. 5A-F).

### Example 3

This example describes that the present experimental vaccine induces Th1-driven immune responses and resistance to Neisseria gonorrhoeae infection in a murine model.

Results

Intravaginal Immunization of Mice with Gonococcal OMV Plus IL-12/Ms Accelerates Clearance of Challenge Infection with N. gonorrhoeae.

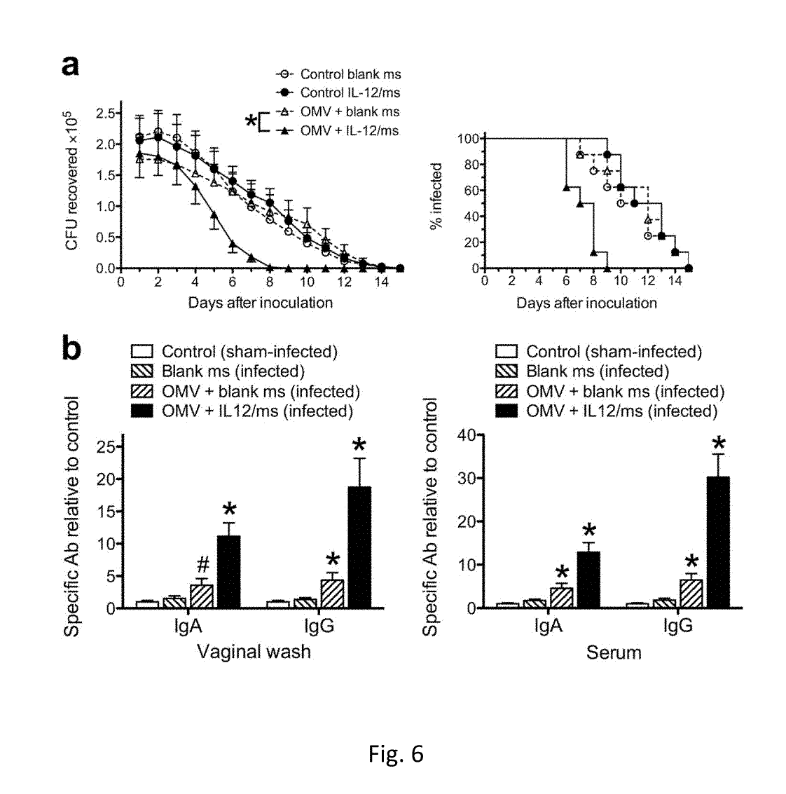

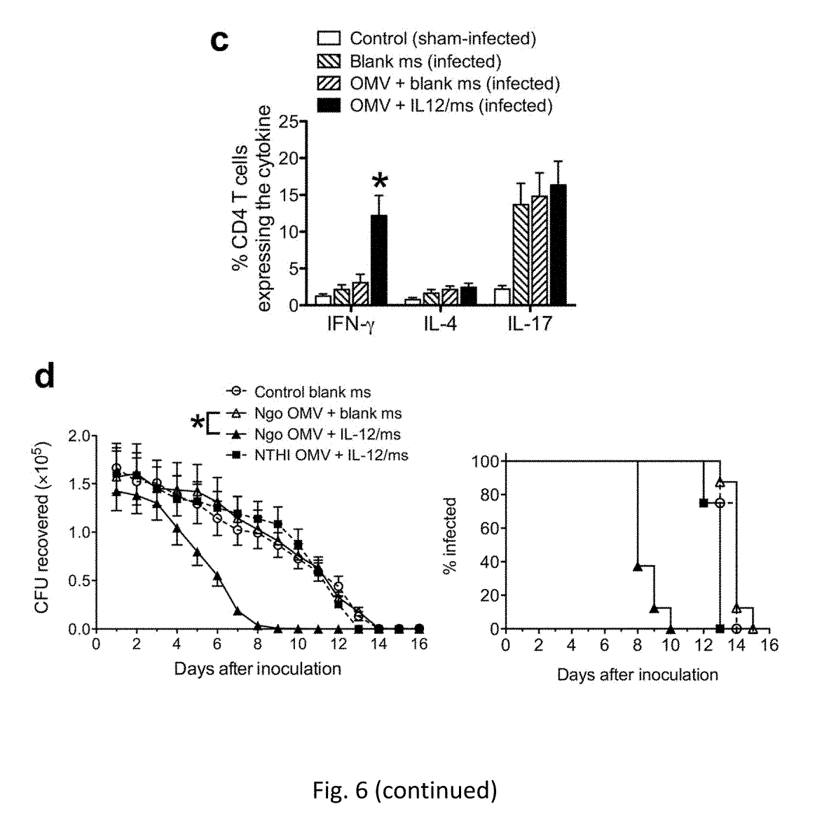

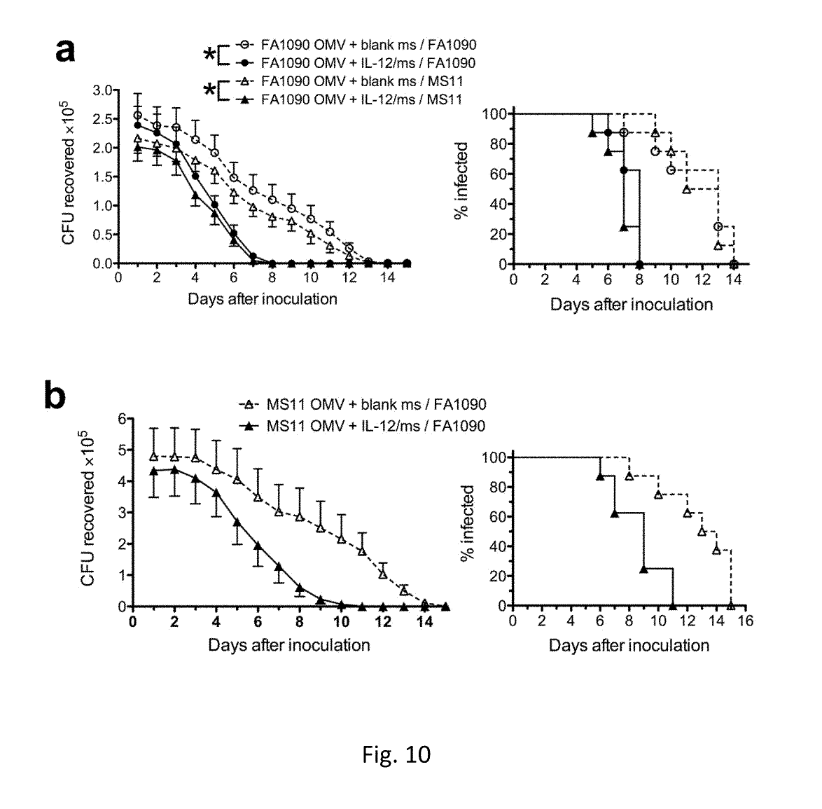

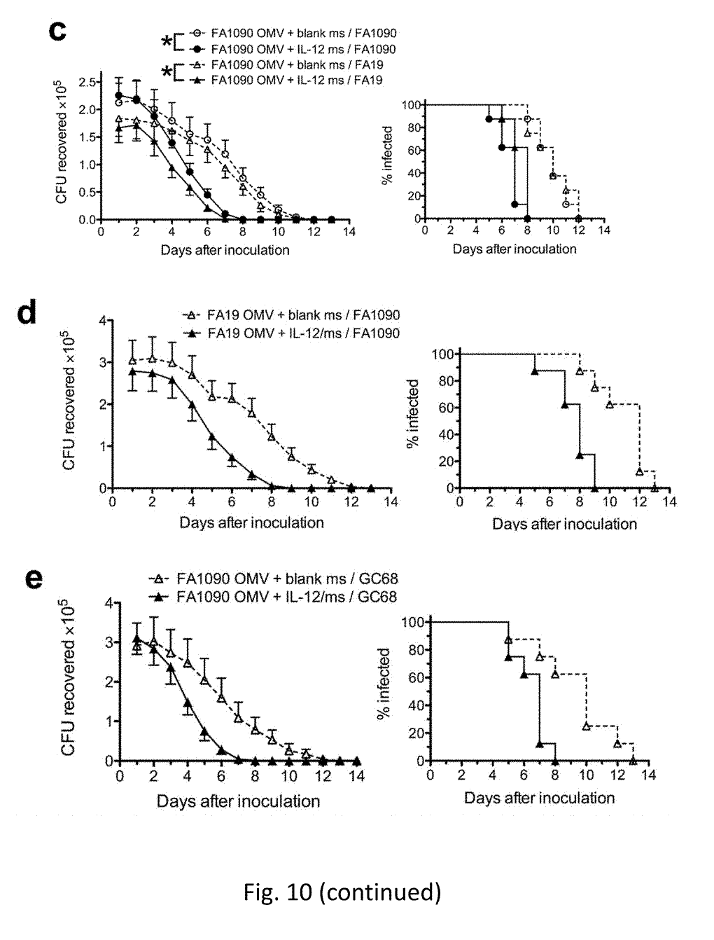

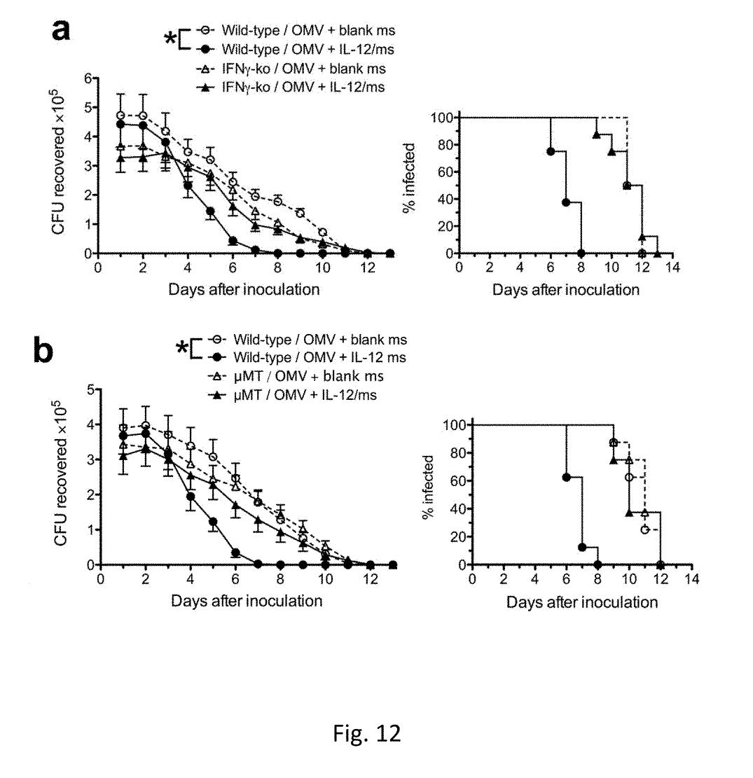

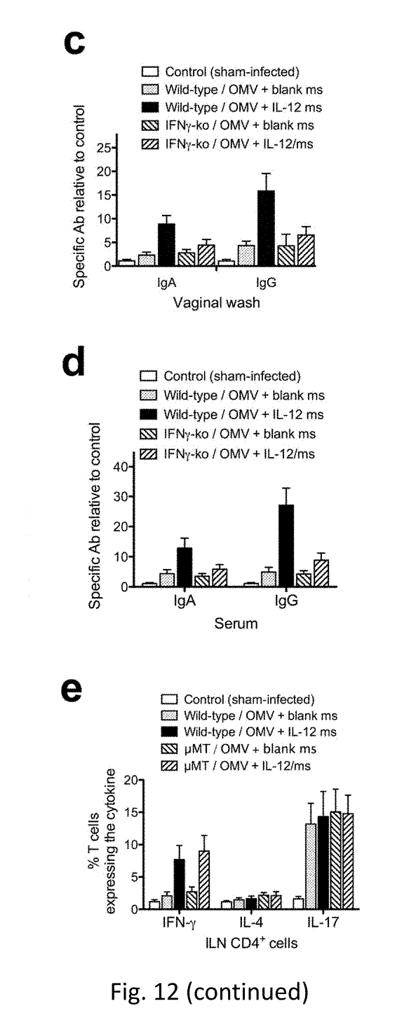

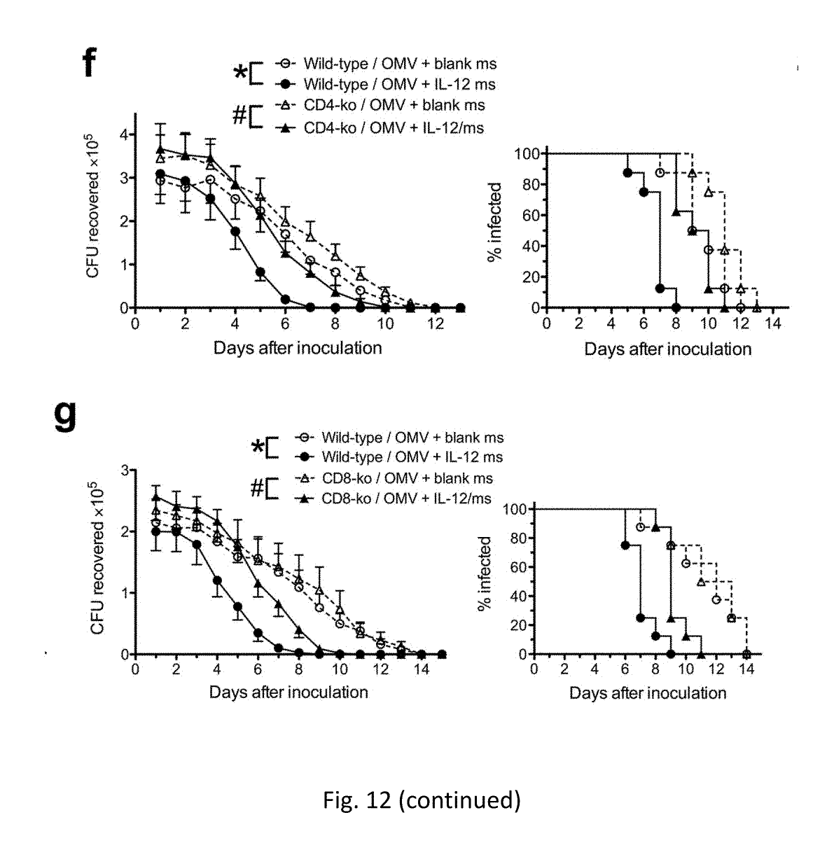

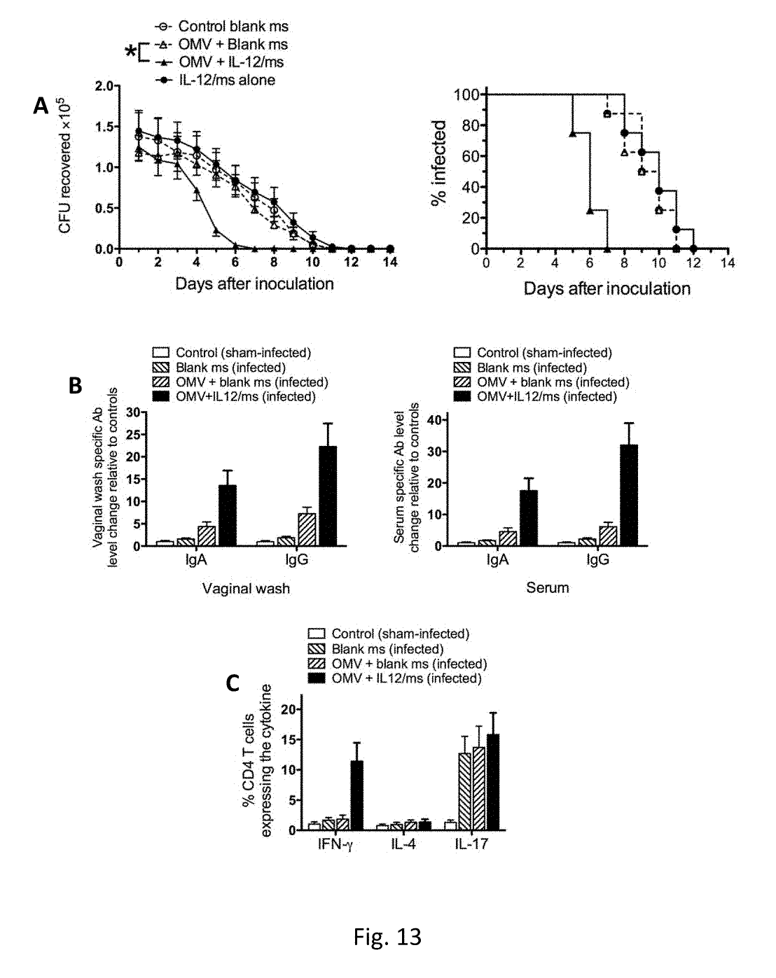

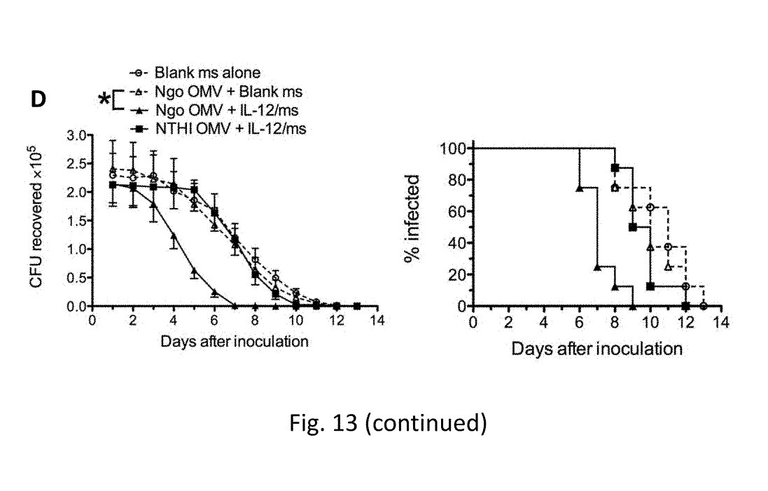

Groups of 8 female BALB/c mice were immunized i.vag. with gonococcal OMV (strain FA1090; 40 μg protein) plus IL-12/ms (1 μg IL-12), or with OMV plus control (blank) ms; two additional control groups were sham-immunized with IL-12/ms or with blank ms alone. Immunizations were repeated 1 week and 2 weeks later, and all mice were challenged after a further 2 weeks by i.vag. instillation of N. gonorrhoeae FA1090 (5×10⁶ CFU). Control (sham-immunized) mice, or mice immunized with OMV plus blank ms cleared the infection commencing at day 7 post-challenge and were all cleared by day 15; median days of clearance were 10-13. There was no significant difference in the clearance rates between these three control groups (FIG. 6a). However, mice immunized with OMV plus IL-12/ms cleared the infection beginning at day 6 and were all cleared by day 9; median clearance=7.5 days compared to 12 days in mice immunized with OMV plus blank ms (P<0.01, Kaplan-Meier; Table 1 and FIG. 6a). This experiment was repeated twice more with similar results (see Table 1 and FIG. 13). Further examples of replication of this finding can be seen in subsequent experiments reported below, e.g., FIGS. 6d, 9, 10a, and c (and FIGS. 10b, d, and e for other gonococcal strains), and FIGS. 12a, b, f, and g (with C57BL/6 mice).

TABLE 1
Summary data from immunization experiments
using homologous challenge (BALB/c mice).

| | | | | | Median | P | |
|---|---|---|---|---|---|---|---|
| | | Vaccine | | Challenge | clearance | (Kaplan- | |
| Expt | Group | OMV strain | Adjuvant | strain | day | Meier) | Notes |
| 1 | a | — | Blank ms | FA1090 | 11 | a - b - c | Data from |
| | b | — | IL-12 ms | FA1090 | 12 | NS | FIG. 6a |
| | c | FA1090 | Blank ms | FA1090 | 12 | <0.01 | |
| | d | FA1090 | IL-12 ms | FA1090 | 7.5 | | |
| 2 | a | — | Blank ms | FA1090 | 9 | a - b - c | Data from |
| | b | — | IL-12 ms | FA1090 | 10 | NS | FIG. 13a |
| | c | FA1090 | Blank ms | FA1090 | 9.5 | <0.001 | |
| | d | FA1090 | IL-12 ms | FA1090 | 6 | | |
| 3 | a | — | Blank ms | FA1090 | 23.5 | a - b - c | Replicate |
| | b | — | IL-12 ms | FA1090 | 24 | NS | of FIG. 6a |
| | c | FA1090 | Blank ms | FA1090 | 22.5 | <0.0001 | |
| | d | FA1090 | IL-12 ms | FA1090 | 10.5 | | |
| 4 | a | — | Blank ms | FA1090 | 14 | a vs. b | Data from |
| | b | FA1090 | Blank ms | FA1090 | 14 | NS | FIG. 6d |
| | c | FA1090 | IL-12 ms | FA1090 | 8 | b vs. c | |
| | | | | | | <0.0001 | |
| | d | NTHI | IL-12 ms | FA1090 | 13 | c vs. d | |
| | | | | | | <0.0001 | |
| 5 | a | — | Blank ms | FA1090 | 11 | a vs. b | Data from |
| | b | FA1090 | Blank ms | FA1090 | 10 | NS | FIG. 13d |
| | c | FA1090 | IL-12 ms | FA1090 | 7 | b vs. c | |
| | | | | | | <0.001 | |
| | d | NTHI | IL-12 ms | FA1090 | 9.5 | c vs. d | |
| | | | | | | <0.001 | |
| 6 | a | — | Blank ms | FA1090 | 9.5 | a vs. b | Data from |
| | | | | | | NS | FIG. 9 |
| | b | FA1090 | Blank ms | FA1090 | 10 | <0.001 | (tested at |
| | c | FA1090 | IL-12 ms | FA1090 | 6.5 | | 6 mos) |
| 7 | a | — | Blank ms | FA1090 | 11.5 | a vs. b | Replicate |
| | | | | | | NS | of FIG. 9 |
| | b | FA1090 | Blank ms | FA1090 | 11 | <0.001 | (tested at |
| | c | FA1090 | IL-12 ms | FA1090 | 7 | | 4 mos) |

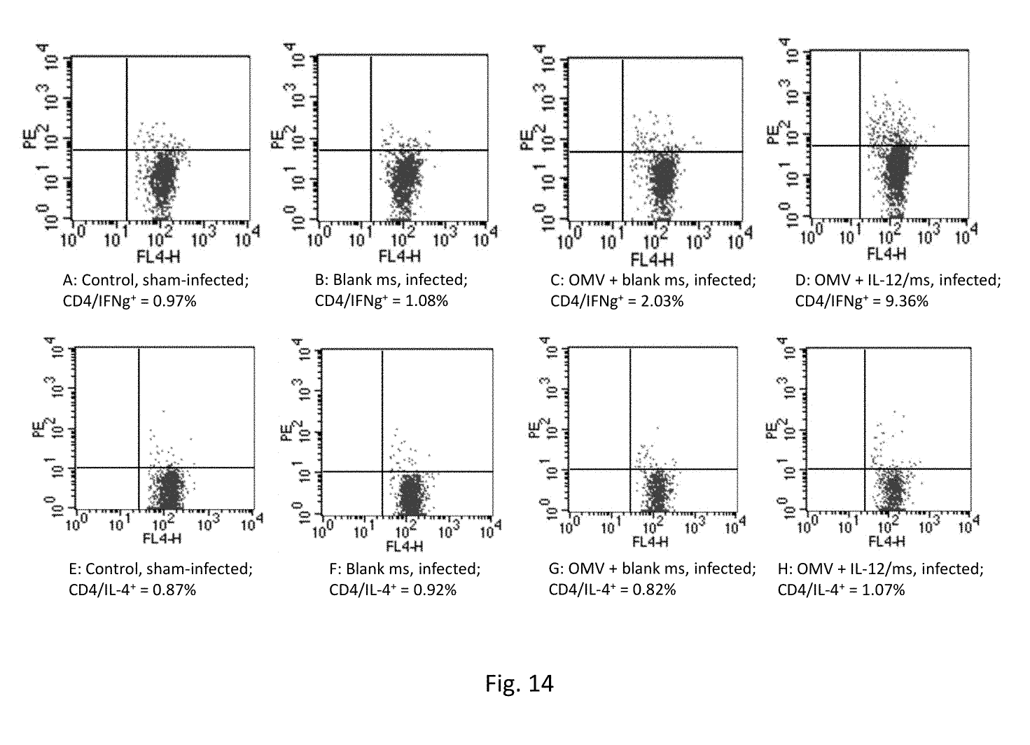

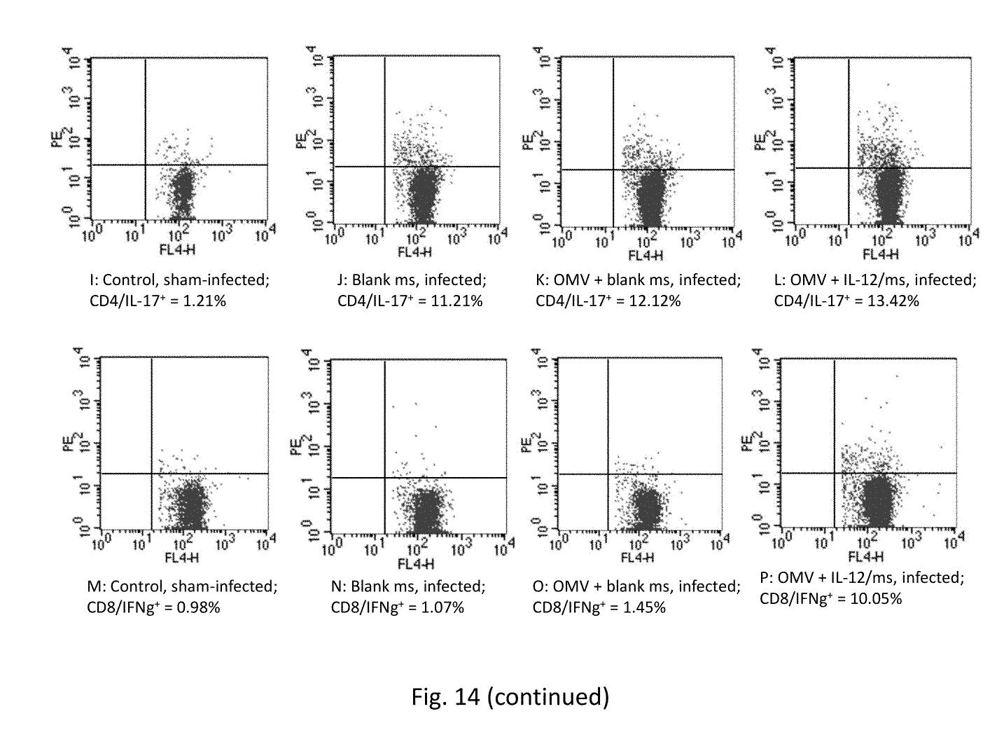

Serum and vaginal wash samples collected after clearance (at termination, day 15 post-inoculation) were assayed for antibodies against intact gonococci (FA1090) by ELISA. This showed that mice immunized with OMV plus IL-12/ms had developed the highest levels of vaginal and serum IgG and IgA antibodies, whereas those mice immunized with OMV plus blank ms developed much lower levels of these antibodies (FIG. 6b). Mice that were unimmunized and sham-infected showed no antibodies detectable above assay background at the starting dilutions, and mice immunized with blank ms alone and infected also did not develop detectable antibodies (FIG. 6b). Iliac lymph node (ILN) mononuclear cells collected at the same time were stained for surface CD4 expression and for intracellular cytokines, and analyzed by flow cytometry. This revealed that only mice immunized with OMV plus IL-12/ms generated CD4⁺/IFNγ⁺ (and CD8⁺/IFNγ⁺) T cells, whereas no mice developed significant numbers of CD4⁺/IL-4⁺ T cells (FIG. 6c; see also FIG. 14). However, all mice that were infected with N. gonorrhoeae regardless of immunization developed CD4⁺/IL-17⁺ T cells (FIG. 6c and FIG. 14).

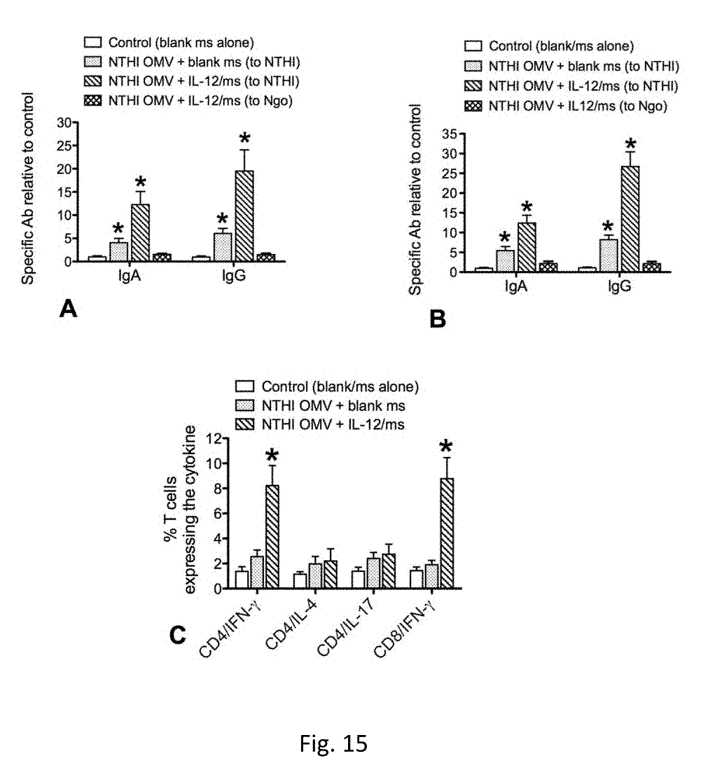

Further experiments were performed to determine the minimum number of immunizations required to induce immune resistance to infection. A single dose of OMV plus IL-12/ms given i.vag. did not consistently generate resistance to challenge, but two doses of OMV (40 μg protein) plus IL-12/ms (1 μg IL-12) given at an interval of 2 weeks were sufficient to induce similar resistance to infection; median clearance=8 days (FIG. 6d; Table 1). In addition, control immunization with OMV prepared from non-typable Haemophilus influenzae (NTHI) plus IL-12/ms failed to induce resistance to N. gonorrhoeae; median clearance=13 days (FIG. 6d; Table 1). This induced antibodies to NTHI but not to N. gonorrhoeae (FIGS. 15a and b) and generated IFNγ-producing CD4⁺ and CD8⁺ cells in the ILN (FIG. 15c).

Intravaginal Immunization with Gonococcal OMV Plus IL-12/Ms Induces Persistent Gonococcus-Specific Antibody Responses and Th1 Cellular Responses.

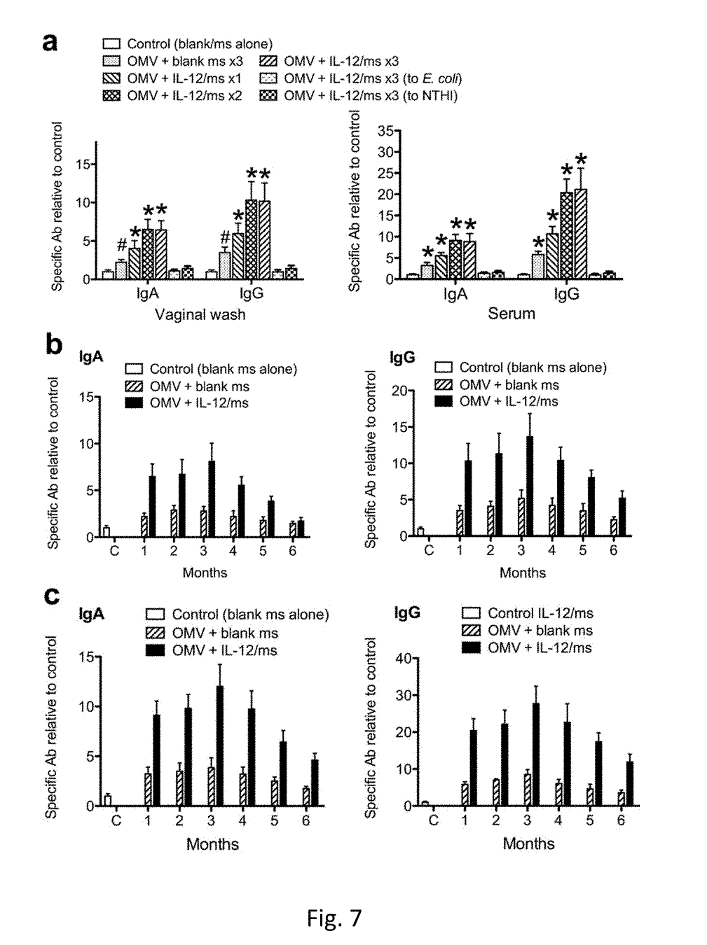

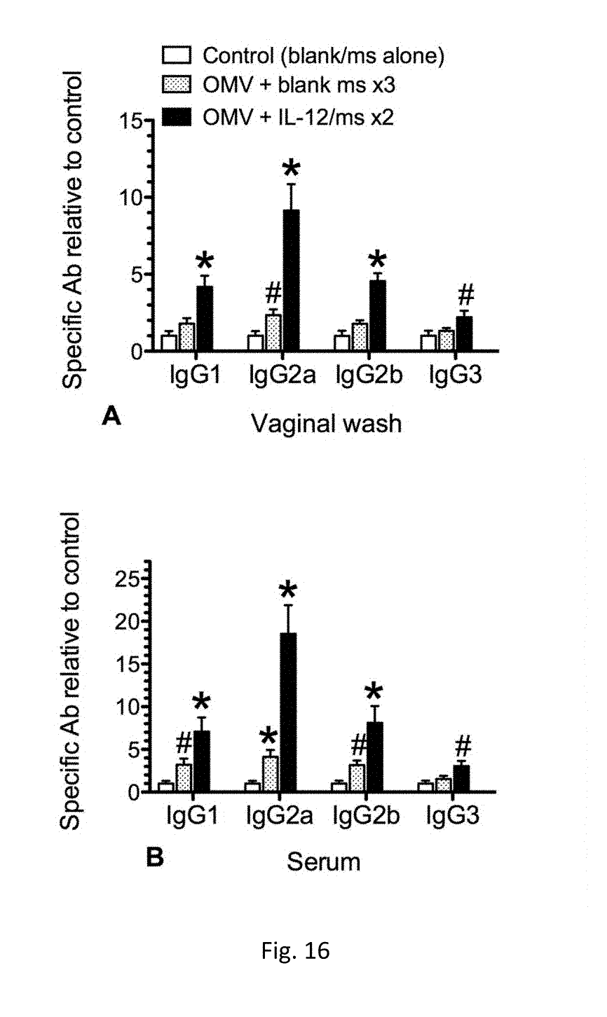

To characterize the local and systemic immune responses after immunization with 1, 2, or 3 doses of gonococcal OMV plus IL-12/ms and before challenge, serum, vaginal washes, and ILN were collected from immunized and control mice 2 weeks after the last immunization. Serum anti-gonococcal IgM antibodies were at low levels with little difference between experimental groups. IgA and IgG antibodies were not detectable above background in vaginal wash or serum samples of mice given blank ms alone. Intravaginal immunization with 3 doses of gonococcal OMV plus blank ms elevated vaginal and serum anti-gonococcal IgA and IgG antibodies but to a lesser degree than immunization with OMV plus IL-12/ms (FIG. 7a). In contrast, immunization with one dose of OMV plus IL-12/ms induced low levels of anti-gonococcal antibodies in both serum and vaginal wash; a second dose elevated antibody production, but no further elevation was seen after 3 doses (FIG. 7a). Antibodies appeared to be specific for N. gonorrhoeae, as they were not detected above control levels against E. coli or NTHI. Assay of IgG subclass antibodies in both vaginal wash and serum showed a predominance of IgG2a, with lesser amounts of IgG1 and IgG2b and low levels of IgG3 (FIG. 16). The production of anti-gonococcal IgA and IgG antibodies in vaginal wash and serum peaked at 3 months after immunization with 2 doses of OMV plus IL-12/ms, and were still detectable at 6 months after immunization (FIGS. 7b and c).

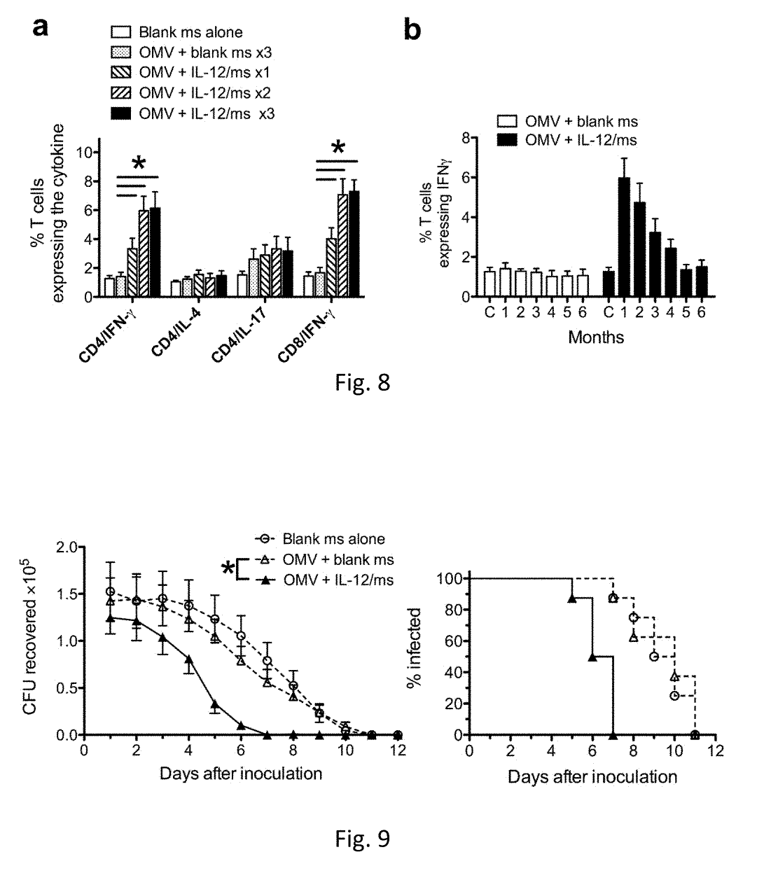

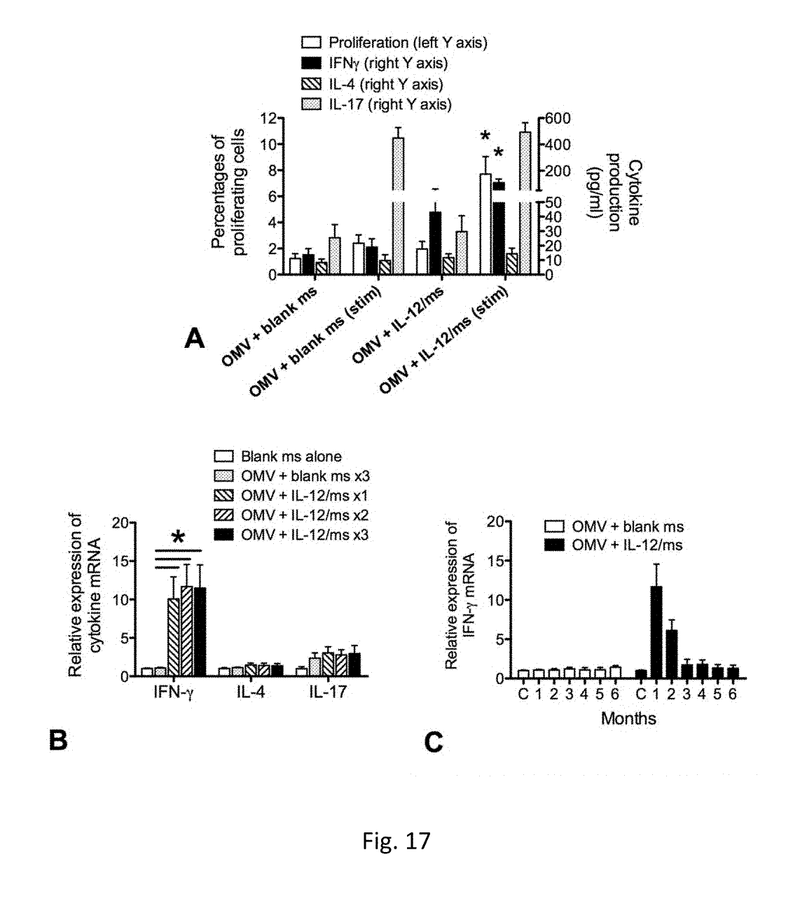

Flow cytometric analysis of ILN cells revealed increased numbers of IFNγ⁺/CD4⁺ and IFNγ⁺/CD8⁺ T cells from mice immunized with OMV plus IL-12/ms compared with those from control mice (FIG. 8a). As observed with the antibody responses, one immunization was sufficient to induce IFNγ production, and it was further elevated by 2 immunizations; 3 doses did not further increase it. In contrast, immunization with OMV plus IL-12/ms did not significantly increase the numbers of IL-4⁺/CD4⁺ and IL-17⁺/CD4⁺ T cells relative to controls (FIG. 8a). To determine whether the induced IFNγ⁺/CD4⁺ (and IFNγ⁺/CD8⁺) T cells were specific for gonococcal antigens, CD4⁺ cells isolated from ILN were preloaded with CFSE, cultured in vitro for 3 days in the presence of antigen-presenting cells with gonococcal OMV or without stimulation as controls, and their proliferation was assessed by flow cytometry. CD4⁺ cells from the ILN of immunized mice proliferated significantly more, and produced significantly more IFNγ, in response to stimulation in vitro, than the cells from control mice (FIG. 17a). No production of IL-4 was seen, but IL-17 was generated by CD4⁺ T cells cultured with gonococcal OMV (FIG. 17a). IFNγ production by ILN CD4⁺ T cells remained elevated, albeit at slowly declining levels, for 4 months after immunization (FIG. 8b).

In addition, vaginas were excised from euthanized mice 3 days after the last immunization and RNA was extracted from the whole tissue. RT-PCR analysis showed that, in comparison to controls, immunization with gonococcal OMV plus IL-12/ms significantly enhanced the expression of mRNA for IFNγ but not for IL-4 or IL-17 (FIG. 17b). IFNγ mRNA expression in vaginal tissue, and production of IFNγ by ILN CD4⁺ cells remained elevated for up to 2 months following i.vag. immunization with gonococcal OMV plus IL-12/ms (FIG. 17c). These findings support the cytokine expression results obtained with cells from the draining ILN.

Duration of Vaccine-Induced Resistance to Infection.

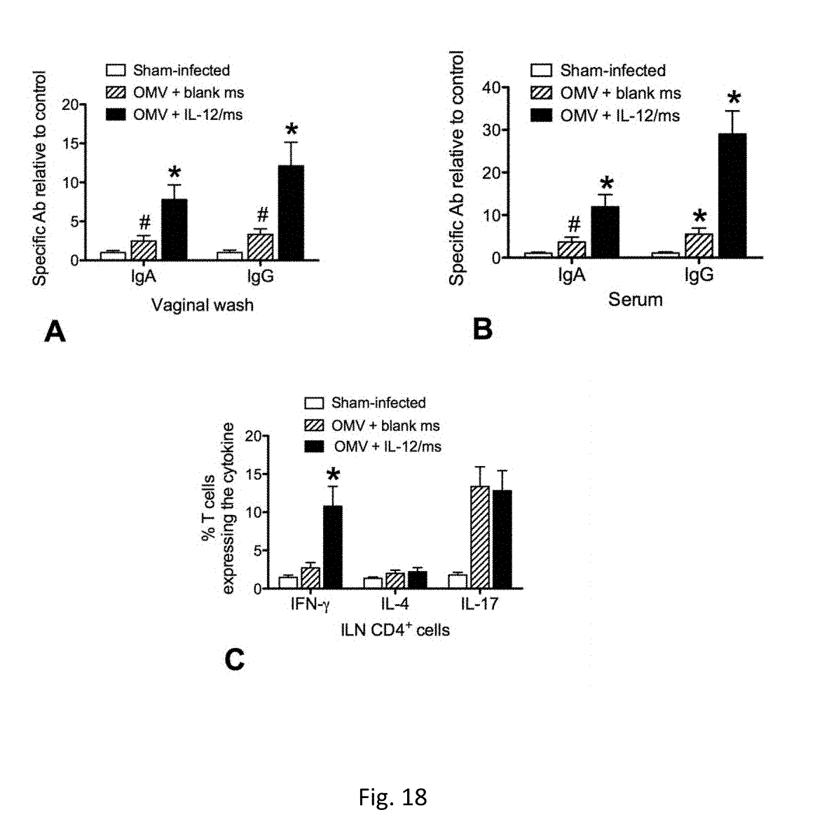

To evaluate the duration of immune resistance, groups of 8 mice were immunized with gonococcal OMV plus IL-12/ms, and were challenged with the same strain (FA1090) of N. gonorrhoeae at 2, 4, or 6 months after immunization. Compared to age-matched controls that were either sham-immunized or immunized with OMV plus blank ms, mice immunized with OMV plus IL-12/ms were resistant to N. gonorrhoeae infection when challenged at 2 or 4 months after immunization; median clearance in controls=11-11.5 days vs. 7 days in immunized mice (Table 1=Suppl. Table 1). Similar results were obtained in a replicate experiment when mice were challenged 6 months after immunization; median clearance in controls=9.5-10 days vs. 6.5 days in mice immunized with OMV plus IL-12/ms (FIG. 9; Table 1). After termination anti-gonococcal antibodies were detected in serum and vaginal washes (FIGS. 18a and b). IFNγ- (but not IL-4-) secreting CD4⁺ T cells were present in ILN (FIG. 18c). Notably, the antibody and IFNγ responses detected after challenge were higher than those before challenge (compare with FIGS. 7b, c, and 8b) implying recall of memory. As observed previously, IL-17-secreting T cells were always found after challenge with N. gonorrhoeae, regardless of immunization (FIG. 18c). Longer time periods were not evaluated because mice become increasingly resistant to gonococcal infection as they age.

Immunization Induces Resistance to Heterologous Strains of N. gonorrhoeae.

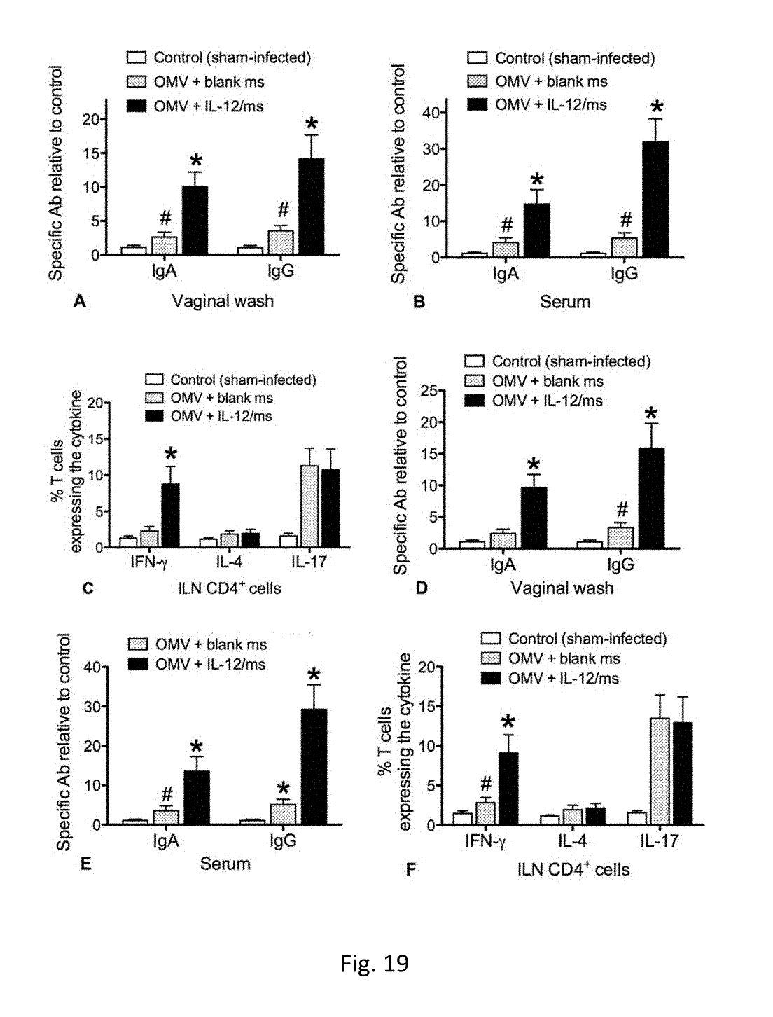

An important consideration for any vaccine is that it should be effective against different strains of the pathogen, as well as those antigenically homologous to the immunizing strain. N. gonorrhoeae is well known for its antigenic variability involving most of its surface antigens through multiple molecular mechanisms. We therefore determined whether i.vag. immunization with one strain of gonococcal OMV would be effective against challenge with other strains to a similar extent as challenge with the same strain. At first, mice (8 per group) were immunized i.vag. with OMV prepared from strain FA1090 plus IL-12/ms or blank ms, and were challenged one month later with N. gonorrhoeae strains FA1090 or MS11 (5×10⁶ CFU). Immunization with FA1090 OMV induced resistance to challenge with either FA1090 or MS11 to similar extents (FIG. 10a; Table 2). After challenge and clearance antibodies were elevated to similar levels against MS11 and the Th1 responses indicated by IFNγ⁺/CD4⁺ T cells in ILN were similarly enhanced (FIGS. 19a, b, and c). In a reciprocal manner, immunization with MS11 OMV (plus IL-12/ms) induced resistance to challenge with strain FA1090 (FIG. 10b; Table 2).

TABLE 2
Summary data from immunization experiments
using heterologous challenge (BALB/c mice).

| | | | | | Median | P | |
|---|---|---|---|---|---|---|---|
| | | Vaccine | | Challenge | clearance | (Kaplan- | |
| Expt | Group | OMV strain | Adjuvant | strain | day | Meier) | Notes |
| 1 | a | FA1090 | Blank ms | FA1090 | 13 | <0.002 | Data from |
| | b | FA1090 | IL-12 ms | FA1090 | 8 | | FIG. 10a |
| | c | FA1090 | Blank ms | MS11 | 12 | <0.0001 | |
| | d | FA1090 | IL-12 ms | MS11 | 7 | | |
| 2 | a | FA1090 | Blank ms | FA1090 | 10.5 | <0.002 | Data from |
| | b | FA1090 | IL-12 ms | FA1090 | 7.5 | | FIG. 10a |
| | c | FA1090 | Blank ms | MS11 | 11 | <0.001 | |
| | d | FA1090 | IL-12 ms | MS11 | 8 | | |
| 3 | a | MS11 | Blank ms | FA1090 | 13.5 | <0.01 | Data from |
| | b | MS11 | IL-12 ms | FA1090 | 9 | | FIG. 10b |
| 4 | a | MS11 | Blank ms | FA1090 | 11 | <0.01 | Data from |
| | b | MS11 | IL-12 ms | FA1090 | 8 | | FIG. 10b |
| 5 | a | FA1090 | Blank ms | FA1090 | 10 | <0.01 | Data from |
| | b | FA1090 | IL-12 ms | FA1090 | 8 | | FIG. 10c |
| | c | FA1090 | Blank ms | FA19 | 10 | <0.0001 | |
| | d | FA1090 | IL-12 ms | FA19 | 7 | | |
| 6 | a | FA1090 | Blank ms | FA1090 | 10 | <0.01 | Data from |
| | b | FA1090 | IL-12 ms | FA1090 | 7 | | FIG. 10c |
| | c | FA1090 | Blank ms | FA19 | 10 | <0.0001 | |
| | d | FA1090 | IL-12 ms | FA19 | 6 | | |
| 7 | a | FA19 | Blank ms | FA1090 | 12 | <0.01 | Data from |
| | b | FA19 | IL-12 ms | FA1090 | 8 | | FIG. 10d |
| 8 | a | FA19 | Blank ms | FA1090 | 9 | <0.01 | Replicate of |
| | b | FA19 | IL-12 ms | FA1090 | 7 | | FIG. 10d |
| 9 | a | FA1090 | Blank ms | GC68 | 10 | <0.01 | Data from |
| | b | FA1090 | IL-12 ms | GC68 | 7 | | FIG. 10e |
| 10 | a | FA1090 | Blank ms | GC68 | 12.5 | <0.001 | Replicate of |
| | b | FA1090 | IL-12 ms | GC68 | 8 | | FIG. 10e |
| 11 | a | FA1090 | Blank ms | GC69 | 12.5 | <0.001 | Additional |
| | | | | | | | clinical |
| | b | FA1090 | IL-12 ms | GC69 | 8 | | isolate |
| 12 | a | FA19 | Blank ms | MS11 | 9 | <0.01 | Additional |
| | | | | | | | heterologous |
| | b | FA19 | IL-12 ms | MS11 | 7 | | challenge |
| 13 | a | MS11 | Blank ms | FA19 | 11 | <0.01 | Reciprocal |
| | b | MS11 | IL-12 ms | FA19 | 8 | | of expt. 12 |

Gonococcal strains FA1090 and MS11 both possess porin of the same major type (PorB.1B) although of different subtypes. Therefore, to determine whether the major porin type is integral to immune resistance, further experiments were performed with strain FA19 (PorB.1A). Immunization with FA1090 OMV (plus IL-12/ms) induced resistance to challenge with FA19 (FIG. 10c; Table 2). Antibody responses assayed at termination showed cross-reactivity against FA19, and IFNγ⁺/CD4⁺ T cells in ILN were elevated (FIGS. 19d, e, and g). Reciprocally, immunization with FA19 OMV induced resistance to challenge with strain FA1090 (FIG. 10d; Table 2). Other immunization and challenge combinations (i.e., MS11 against FA19, and vice versa) likewise showed similar cross-resistance (Table 2).

N. gonorrhoeae strains FA1090, MS11, and FA19 have been widely used in many laboratories and extensively subcultured since their original isolation. As a result, it is possible that they have acquired mutations and become altered in some of their characteristics. Therefore we also challenged immunized mice with novel clinical strains that have been minimally passaged in vitro since their isolation. Mice immunized with FA1090 OMV plus IL-12/ms were also resistant to challenge with clinical isolates GC68 (a PorB.1B strain; FIG. 10e; Table 2) and GC69 (PorB.1A; Table 2).

Antigens Targeted by Immunization-Induced Antibodies.

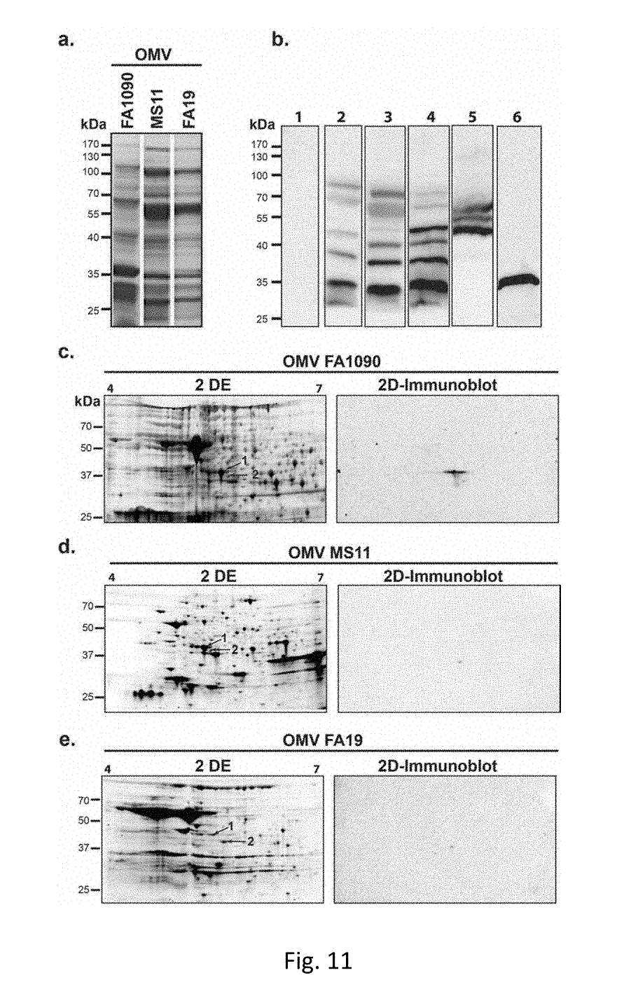

When examined by one-dimensional (1D) SDS-PAGE, the protein profiles of FA1090, MS11, and FA19 OMV were similar, but with apparent quantitative as well as qualitative variations (FIG. 11a). Western blot analyses of serum from one mouse (#1) immunized with FA1090 OMV plus IL-12/ms against FA1090, MS11 or FA19 OMV separated by SDS-PAGE revealed IgG antibodies reactive with bands migrating at approximately 35-80 kDa, with reactivity against bands present in OMV from all three strains strains (FIG. 11b, lanes 2-4). One of these bands at approximately 35 kDa may correspond to porin, as H5 antibody reacted with a band of similar mobility (FIG. 11b, lane 6). Another serum (#2) displayed strong reactivity against three bands migrating at approximately 45-65 kDa (FIG. 11b, lane 5). In an effort to identify the ˜45-65-kDa antigens, we used immunoproteomic approaches consisting of two-dimensional (2D) SDS-PAGE separation of OMV and parallel 2D SDS-PAGE followed by immunoblotting (2D-immunoblot) and mass spectrometry. The three 2D protein maps of OMV revealed by Flamingo staining showed numerous protein species and significant differences in the OMV proteome between FA1090, MS11, and FA19 strains (FIGS. 11c, d, and e). In contrast, the blotted protein maps showed two spots (Spot 1 and Spot 2) of masses corresponding to 45 kDa and 43 kDa, and pI 5.2 and 5.5, respectively (FA1090 OMV; FIG. 11c), or one spot (Spot 1) with an approximate mass 45 kDa and pI 5.2 (MS11 and FA19 OMV; FIGS. 11d and e). Mass spectrometry analysis of the tryptic peptides obtained from Spot 1 and Spot 2 (FIGS. 11c, d, and e) revealed as top hits translation elongation factor-Tu (EF-Tu) and a putative periplasmic polyamine-binding protein, PotF3, respectively (Table 3). EF-Tu appeared as the most confident antigen as it was immunoreactive in all three 2D-immunoblots and was identified with the highest confidence (score ranging from 485.0 to 947.1) and coverage (64.2 to 90.6) in all OMV preparations (Table 3).

TABLE 3
Protein identification of 2D-SDS-PAGE gel spots from gonococcal OMV (Spot 1 and Spot 2; shown
in FIGS. 11c-e) recognized by sera from mice immunized with FA1090 OMVs plus IL-12/ms.

| | | | | | | | | |
|---|---|---|---|---|---|---|---|---|
| | | | | | | MW | calc. | |
| Strain | Accession | Description | Gene | Score | Coverage | [kDa] | pl | Localization |

Spot 1

| | | | | | | | | |
|---|---|---|---|---|---|---|---|---|
| FA1090 | Q5F5Q8, | elongation factor Tu | tuf1, NGO1842 | 583.1 | 71.8 | 42.91 | 5.30 | Cytoplasmic/¹ |
| | YP_208891 | | tuf2, NGO1858 | | | | | Periplasmic |
| | YP_207710 | pyruvate | aceE, NGO0565 | 159.8 | 27.1 | 99.53 | 5.80 | Cytoplasmic |
| | | dehydrogenase | | | | | | |
| | | subunit E1 | | | | | | |
| | YP_209108 | molecular chaperone | groL groEL, NGO2095 | 101.9 | 38.1 | 57.31 | 5.03 | Cytoplasmic |
| | | GroEL | | | | | | |
| | YP_207230 | carbamoyl phosphate | carA, NGO0053 | 83.6 | 43.8 | 40.60 | 5.43 | Cytoplasmic |
| | | synthase small | | | | | | |
| | | subunit | | | | | | |
| | YP_208577 | cell division | ftsA, NGO1529 | 77.6 | 54.4 | 44.03 | 5.52 | Inner |
| | | protein FtsA | | | | | | membrane |
| FA19 | EEZ46817 | elongation factor | tuf1, tuf2, NGEG_02139, | 485.0 | 64.2 | 42.91 | 5.30 | Cytoplasmic/¹ |
| | | Tu | NGO1842, NGO1858 | | | | | Periplasmic |
| | EEZ44759 | molecular | NGEG_00029, NGO2095 | 242.0 | 46.9 | 57.30 | 5.03 | Cytoplasmic |
| | | chaperone GroEL | | | | | | |
| | EEZ44814 | ATP synthase F0F1 | NGEG_00084, NGO1205 | 143.5 | 45.4 | 50.39 | 5.16 | Unknown |
| | | subunit beta | | | | | | |
| | EEZ45398 | pyruvate | aceE, NGEG_00668, | 109.6 | 25.0 | 99.53 | 5.80 | Cytoplasmic |
| | | dehydrogenase | NGO0565 | | | | | |
| | EEZ44683 | FKBP-type peptidyl- | fkpA, NGEG_01946, | 109.5 | 44.1 | 28.94 | 5.86 | Outer |
| | | prolyl cis-trans | NGO1225 | | | | | membrane |
| | | isomerase FkpA | | | | | | |
| MS11 | AGU85180, | elongation factor | tuf1, tuf2, NGFG_02465, | 947.1 | 90.6 | 42.91 | 5.30 | Cytoplasmic/¹ |
| | AGU85181 | Tu | NGFG_02466, NGO1842, | | | | | Periplasmic |
| | | | NGO1858 | | | | | |
| | EEZ48906 | signal recognition | NGFG_01822, | 94.5 | 44.4 | 44.30 | 5.22 | Inner |
| | | particle-docking | NGO2060 | | | | | membrane |
| | | protein FtsY | | | | | | |
| | EEZ48268 | polyamine ABC | potF3, NGFG_01435, | 89.4 | 45.5 | 41.29 | 5.96 | Periplasmic |
| | | transporter substrate- | NGO1494 | | | | | |
| | | binding protein | | | | | | |
| | EEZ47020 | carbamoyl-phosphate | carA, NGFG_00187, | 87.9 | 38.5 | 40.62 | 5.43 | Cytoplasmic |
| | | synthase | NGO0053 | | | | | |
| | EEZ47069 | pilus assembly | NGFG_00236, | 78.5 | 43.4 | 41.23 | 5.36 | Unknown |
| | | protein PilM | NGO0098 | | | | | |

Spot 2

| | | | | | | | | |
|---|---|---|---|---|---|---|---|---|
| FA1090 | YP_208544 | ABC transporter | potF3, NGO1494 | 167.5 | 43.7 | 41.23 | 5.96 | Periplasmic |
| | | periplasmic | | | | | | |
| | | binding protein, | | | | | | |
| | | polyamine | | | | | | |
| | YP_207371 | ABC transporter | potF1, NGO0206 | 70.7 | 27.3 | 41.16 | 5.87 | Periplasmic |
| | | periplasmic | | | | | | |
| | | binding protein | | | | | | |
| | | polyamine | | | | | | |
| | YP_207230 | carbamoyl | carA, NGO0053 | 62.6 | 40.1 | 40.60 | 5.43 | Cytoplasmic |
| | | phosphate | | | | | | |
| | | synthase small | | | | | | |
| | | subunit | | | | | | |
| | Q5F5Q8, | elongation factor | tuf1, NGO1842 | 55.5 | 35.3 | 42.91 | 5.30 | Cytoplasmic |
| | YP_208891 | Tu | tuf2, NGO1858 | | | | | |
| | YP_208021 | succinyl-CoA | sucC, NGO0913 | 42.7 | 30.7 | 41.28 | 5.44 | Cytoplasmic |
| | | synthetase | | | | | | |
| | | subunit beta | | | | | | |
| FA19 | EEZ46094 | hypothetical | potF3, NGEG_01364, | 348.1 | 55.5 | 41.26 | 5.96 | Periplasmic |
| | | protein | NGO1494 | | | | | |
| | EEZ45054 | putrescine | potF1, NGEG_00324, | 152.9 | 43.8 | 41.23 | 5.87 | Periplasmic |
| | | transport system | NGO0206 | | | | | |
| | | substrate-binding | | | | | | |
| | | protein | | | | | | |
| | EEZ45398 | pyruvate | aceE, NGEG_00668, | 146.0 | 32.0 | 99.53 | 5.80 | Cytoplasmic |
| | | dehydrogenase | NGO0565 | | | | | |
| | EEZ45783 | succinyl-CoA | sucC, NGEG_01053, | 142.7 | 47.9 | 41.27 | 5.35 | Cytoplasmic |
| | | ligase [ADP- | NGO0913 | | | | | |
| | | forming] subunit | | | | | | |
| | | beta | | | | | | |
| | EEZ44847 | DNA polymerase | dnaN, NGEG_00117, | 102.1 | 55.9 | 40.84 | 5.31 | Cytoplasmic |
| | | III, beta subunit | NGO0002 | | | | | |
| MS11 | EEZ48268 | polyamine ABC | potF3, NGFG_01435, | 794.15 | 74.0 | 41.29 | 5.96 | Periplasmic |
| | | transporter | NGO1494 | | | | | |
| | | substrate-binding | | | | | | |
| | | protein | | | | | | |
| | EEZ47174 | putrescine ABC | potF1, NGFG_00341, | 293.78 | 50.4 | 41.20 | 5.87 | Periplasmic |
| | | transporter | NGO0206 | | | | | |
| | | substrate-binding | | | | | | |
| | | protein | | | | | | |
| | EEZ47069 | pilus assembly | NGFG_00236, NGO0098 | 131.0 | 48.3 | 41.23 | 5.36 | Unknown |
| | | protein PilM | | | | | | |
| | AGU85180, | elongation factor | tuf1, tuf2, NGFG_02465, | 110.6 | 58.6 | 42.91 | 5.30 | Cytoplasmic/ |
| | AGU85181 | Tu | NGFG_02466, NGO1842, | | | | | Periplasmmic¹ |
| | | | NGO1858 | | | | | |
| | EEZ48160 | transaldolase | NGFG_01327, NGO1610 | 101.2 | 55.0 | 37.44 | 5.45 | Cytoplasmic |

¹Localization determined by Porcella et. al. 1996 (ref. 45).

Immune Resistance to N. gonorrhoeae Depends on IFNγ and Antibody.

To determine whether the protective effect of immunization with OMV adjuvanted with IL-12/ms is dependent on IFNγ or antibody responses, or on immunity governed by CD4⁺ or CD8⁺ T cells, we performed immunization experiments using mutant C57BL/6 mice deficient in IFNγ (IFNγ-ko) B cells (μMT), CD4⁺ T cells (CD4-ko), or CD8⁺ T cells (CD8-ko). Groups of 8 C57BL/6 wild-type (control) and immumnodeficient mice were immunized with FA1090 OMV plus IL-12/ms or blank ms, and challenged with N. gonorrhoeae FA1090 (5×10⁶ CFU) one month later. The course of vaginal gonococcal infection was not altered in unimmunized immunodeficient mice relative to wild-type controls. All wild-type and immunodeficient mice started to reduce the recoverable gonococcal load from day 7-11 and had cleared the infection by day 12-14 (median 9-13 days), similar to BALB/c mice used in experiments described in the previous sections (FIGS. 12a, b, f, and g; Table 4).

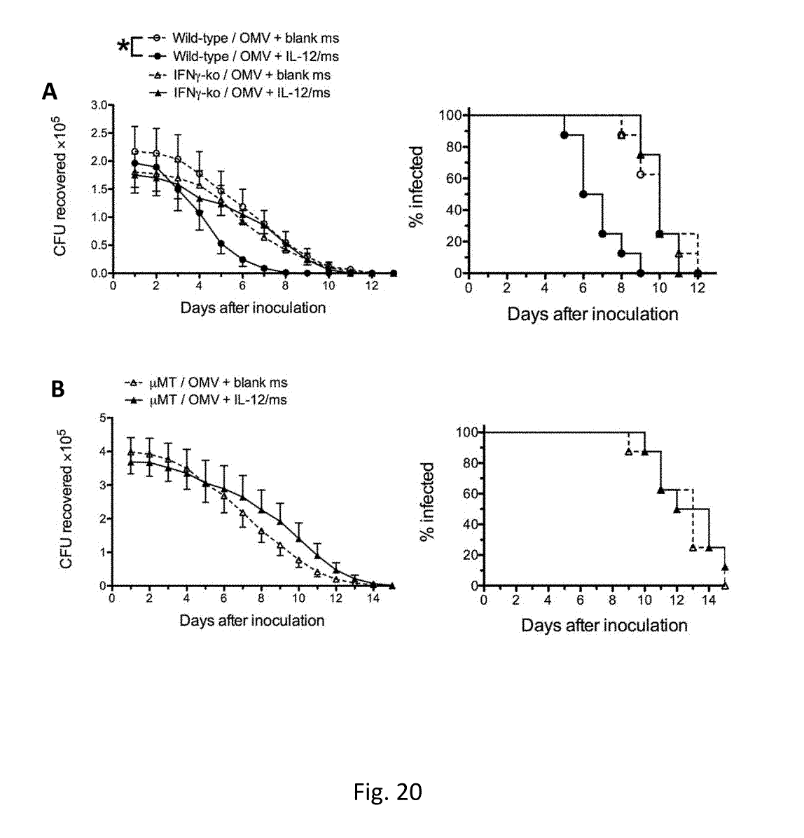

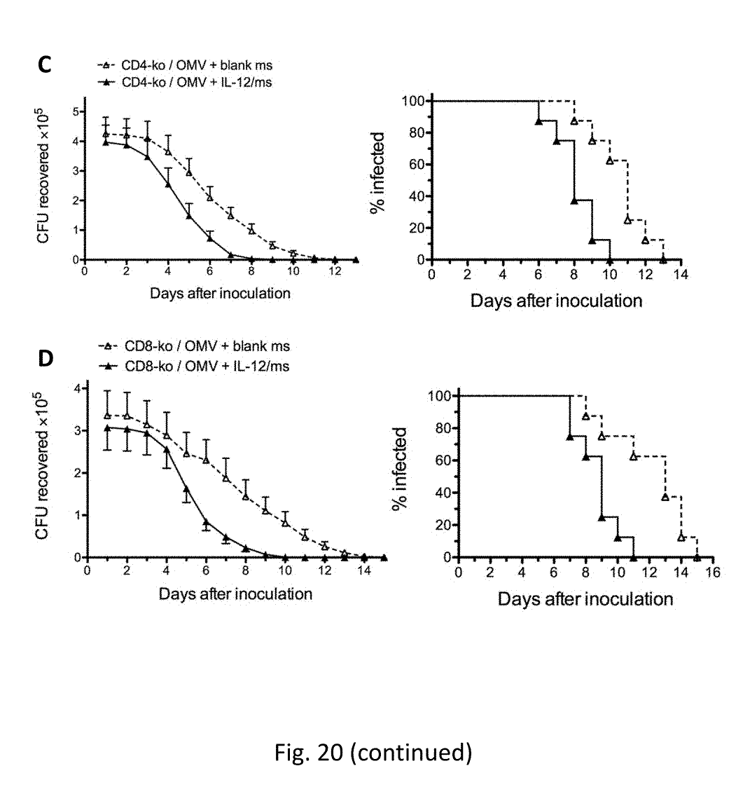

TABLE 4
Summary data from immunization experiments using
immunodeficient mice (C57BL/6 background)

| | | | | | | Median | P | |
|---|---|---|---|---|---|---|---|---|
| | | Mouse | Vaccine | | Challenge | clearance | (Kaplan- | |
| Expt | Group | strain | OMV strain | Adjuvant | strain | day | Meier) | Notes |
| 1 | a | WT | FA1090 | Blank ms | FA1090 | 11.5 | <0.0001 | Data from |
| | b | WT | FA1090 | IL-12 ms | FA1090 | 7 | | FIG. 12a |
| | c | IFNγ-ko | FA1090 | Blank ms | FA1090 | 11.5 | NS | |
| | d | IFNγ-ko | FA1090 | IL-12 ms | FA1090 | 11.5 | | |
| 2 | a | WT | FA1090 | Blank ms | FA1090 | 11 | <0.0001 | Data from |
| | b | WT | FA1090 | IL-12 ms | FA1090 | 7 | | FIG. 12b |
| | c | μMT | FA1090 | Blank ms | FA1090 | 11 | NS | |
| | d | μMT | FA1090 | IL-12 ms | FA1090 | 10 | | |
| 3 | a | WT | FA1090 | Blank ms | FA1090 | 9.5 | <0.001 | Data from |
| | b | WT | FA1090 | IL-12 ms | FA1090 | 7 | | FIG. 12f |
| | c | CD4-ko | FA1090 | Blank ms | FA1090 | 11 | <0.01 | |
| | d | CD4-ko | FA1090 | IL-12 ms | FA1090 | 9.5 | | |
| 4 | a | WT | FA1090 | Blank ms | FA1090 | 12 | <0.001 | Data from |
| | b | WT | FA1090 | IL-12 ms | FA1090 | 7 | | FIG. 12g |
| | c | CD8-ko | FA1090 | Blank ms | FA1090 | 12 | <0.02 | |
| | d | CD8-ko | FA1090 | IL-12 ms | FA1090 | 9 | | |
| 5A | a | WT | FA1090 | Blank ms | FA1090 | 10 | <0.001 | Data from |
| | b | WT | FA1090 | IL-12 ms | FA1090 | 6.5 | | FIG. 20a |
| | c | IFNγ-ko | FA1090 | Blank ms | FA1090 | 10 | NS | |
| | d | IFNγ-ko | FA1090 | IL-12 ms | FA1090 | 10 | | |
| 6 | c | μMT | FA1090 | Blank ms | FA1090 | 13 | NS | Data from |
| | d | μMT | FA1090 | IL-12 ms | FA1090 | 13 | | FIG. 20b |
| 7 | c | CD4-ko | FA1090 | Blank ms | FA1090 | 11 | <0.01 | Data from |
| | d | CD4-ko | FA1090 | IL-12 ms | FA1090 | 8 | | FIG. 20c |
| 8 | c | CD8-ko | FA1090 | Blank ms | FA1090 | 13 | <0.01 | Data from |
| | d | CD8-ko | FA1090 | IL-12 ms | FA1090 | 9 | | FIG. 20d |

In contrast to wild-type mice, clearance of gonococcal infection was not accelerated in IFNγ-ko or μMT mice immunized with OMV plus IL-12/ms compared to immunization with OMV plus blank ms (FIGS. 12a and b; Table 4; FIGS. 20a and b). Thus deficiency of either IFNγ or B cells abrogated the adjuvant effect of IL-12/ms in generating immune resistance to genital gonococcal infection. The production of gonococcus-specific vaginal and serum IgA and IgG antibodies induced by OMV plus IL-12/ms in wild-type mice was abrogated in IFNγ-ko mice (FIGS. 12c and d), and as expected there was no generation of IFNγ by the ILN cells of immunized IFNγ-ko mice (not shown). Likewise, in μMT mice there was no detectable antibody response to immunization (not shown). In contrast, the numbers of IFNγ⁺/CD4⁺ T cells in ILNs of μMT mice immunized with gonococcal OMV plus IL-12/ms were not affected, and there was no IL-4 response, while IL-17 responses remained unaltered (FIG. 10e=FIG. 5e). These findings indicate that resistance induced by immunization with gonococcal OMV plus IL-12/ms depended on both IFNγ and B cells, the latter presumably to produce gonococcus-specific antibodies.

The protective effect of immunization with gonococcal OMV plus IL-12/ms was incompletely diminished in CD4-ko, and partially diminished also in CD8-ko mice, in comparison with wild-type controls (FIGS. 12f and g; Table 4; FIGS. 20c and d. These findings suggest that the requirement for CD4⁺ T cells to generate immune resistance could be partially compensated by other cells, including CD8⁺ or NK cells, which can also produce IFNγ. However, CD8⁺ cells appeared to be less critical for protective immunity.

### Discussion

We have demonstrated for the first time that a vaccine-induced state of immune resistance to genital gonococcal infection can be reliably generated by an intact mammalian immune system. This state of immunity appears to depend on antibody production by B cells, and on the generation of IFNγ mainly by CD4⁺ T cells. I.vag. vaccination of mice with gonococcal OMV plus IL-12/ms as an adjuvant induced serum and vaginal IgG and IgA antibodies against gonococcal antigens, and IFNγ-secreting CD4⁺ and CD8⁺ T cells in the draining ILN. Both Th1 cellular and antibody responses persisted for several months after immunization, and were capable of eliciting resistance to challenge with N. gonorrhoeae for at least 6 months, with the recall of immune memory. I.vag. immunization with gonococcal OMV alone, either without adjuvant or with control (blank) ms, induced only weak antibody responses with no detectable IFNγ production, and no significant resistance to challenge infection. Control immunization with OMV prepared from NTHI plus IL-12/ms did not generate immune resistance or antibodies cross-reactive with N. gonorrhoeae, although an IFNγ response was induced. Thus, while IFNγ appears to be necessary for resistance to N. gonorrhoeae, without specific antibodies it is not sufficient.

We report here that IL-12/ms, given i.vag as an adjuvant with gonococcal OMV vaccine, enhances Th1-driven protective immunity revealed by a significantly shortened course of genital gonococcal infection. It should be emphasized that free soluble IL-12 is ineffective and and that IL-12 encapsulated in ms were required for the adjuvant effect with the OMVs.

Given the well-known and extensive antigenic variation shown by N. gonorrhoeae, resistance extended to heterologous strains as well as against the homologous strain from which the OMV vaccine was prepared ws unexpected. Our results show that immunization with OMV derived from strain FA1090 enhances resistance equally well against strains MS11 and FA19, and vice versa, and that resistance extends to clinical isolates of N. gonorrhoeae in addition to these “laboratory strains”. Among the major gonococcal surface antigens, we know that FA1090, MS11, and FA19 differ in their porin (PorB) molecules. FA1090 and MS11 possess different subtypes of PorB.1B, and FA19 has PorB.1A (Elkins et al., Mol. Microbiol. 14, 1059-1075 (1994)). Although not as well characterized, the Opa proteins encoded in their genomes differ (Hobbs et al., Front. Microbiol. 2, 123 (2011), Cole et al., PLoS One 4, e8108 (2009), and their LOS are different (Erwin et al., J. Exp. Med. 184, 1233-1241 (1996)). Opa proteins and LOS glycan chains are also phase-variable, resulting in the expression of different antigenic epitopes (Apicella, M. A. et al. Phenotypic variation in epitope expression of the Neisseria gonorrhoeae lipooligosaccharide. Infect. Immun. 55, 1755-1761 (1987)).

Consistent with cross-protective immunity, ELISA analysis of antibodies induced by immunization revealed quantitatively similar levels of antibodies detectable against the different strains, with respect to both IgG and IgA in serum and vaginal washes. The antibodies appeared to be specific for N. gonorrhoeae as they were not detected against E. coli or NTHI, and they were not generated by immunization with OMV prepared from NTHI. Western blot analysis of serum IgG antibodies, however, revealed evidence of antigens shared between different strains of N. gonorrhoeae. Bands migrating at 45-65 kDa migrated at higher molecular mass than major gonococcal outer membrane proteins such as porin and Opa which are in the range 30-40 kDa.

Our studies have identified two novel gonococcal vaccine candidates, EF-Tu in FA1090, MS11, and FA19 OMV, and PotF3 also in FA1090. Both proteins have been identified in quantitative proteomic profiling of cell envelopes and OMV derived from four common gonococcal isolates (Zielke et al., Molec. Cell. Proteomics 13, 1299-1317 (2014)). EF-Tu is of particular interest as it has been identified in both spots and in all analyzed OMV. EF-Tu is commonly perceived as a cytosolic GTP-binding protein and an essential factor in protein synthesis.

The present disclosure provides demonstration that individuals can be immunized against N. gonorrhoeae by the i.vag. administration of a non-living vaccine (OMV) with a Th1-driving adjuvant, IL-12/ms. These findings demonstrate the feasibility of a vaccine against N. gonorrhoeae despite previous setbacks and these findings also shed light on the type of immune responses that needs to be induced to generate protective immunity.

### Methods

Mice.

All mice, including wild-type BALB/c and C57BL/6 mice, B6.129S7-Ifng^{tmlTs}/J (IFNγ-deficient), B6.129S2-Ighm^{tmlCgn}/J (B cell-deficient; also known as μMT), B6.129S2-Cd4^{tmlMak}/J (CD4-deficient), and B6.129S2-Cd8a^{tmlMak}/J (CD8-deficient) mice on a C57BL/6 background, were purchased from Jackson Laboratories (Bar Harbor, Me.). BALB/c mice were used for the experiments unless otherwise specified. Mice were maintained in a BSL2 facility in the Laboratory Animal Facility at the University at Buffalo, which is fully accredited by AAALAC. All animal use protocols were approved by the Institutional Animal Care and Use Committee of the University at Buffalo.

Bacteria.

N. gonorrhoeae strain FA1090 was provided by Dr Janne Cannon (University of North Carolina at Chapel Hill); strain MS11 was provided by Dr Daniel Stein (University of Maryland); strain FA19, and clinical isolates were obtained from the collection of clinical strains maintained at the University of North Carolina at Chapel Hill. For use in the murine infection model, N. gonorrhoeae strains 9087 and 0336 were transformed with the streptomycin-resistant rpsL gene from strain FA1090 to generate strains GC68 and GC69, respectively. E. coli K12 was provided by Dr Terry Connell (University at Buffalo). Non-typeable Haemophilus influenzae (NTHI) strain 6P24H1 was provided by Dr Timothy Murphy (University at Buffalo). N. gonorrhoeae was cultured on GC agar supplemented with hemoglobin and ISOVITALEX®, an enrichment medium (BD Diagnostic Systems, Franklin Lakes, N.J.) and the resultant growth was checked for colony morphology consistent with Opa protein and pilus expression. NTHI was cultured on GC agar supplemented with hemoglobin only. E. coli was cultured on BHI agar. Bacteria were harvested from plates and the cell density was determined (Liu et al., Mucosal Immunol. 5, 320-331 (2012)).

IL-12 Microspheres.

Murine IL-12 (Wyeth, Philadelphia, Pa.) was encapsulated into poly-lactic acid microspheres using the Phase Inversion Nanoencapsulation technology as previously described except that bovine serum albumin was replaced by sucrose (0.1%, w/w) (Egilmez et al., Methods Mol. Med. 75, 687-696 (2003)). Blank microspheres were prepared in the same way but without IL-12.

Gonococcal Outer Membrane Vesicles (OMV).

After 18-22 h culture on supplemented GC agar, N. gonorrhoeae was harvested from plates into ice-cold lithium acetate buffer (pH 5.8) and passed through a 25-gauge needle 10-12 times to sheer the outer membranes from the bacteria. The suspensions were spun in microfuge tubes at 13,000 RPM for 1 min. The supernatants were collected and ultracentrifuged at 107,000×g for 2 h. The pellet was washed with 50 mM Tris-HCl (pH 8.0) and resuspended in PBS. Protein was assayed with the Micro BCA protein kit (Thermo Scientific, Rockford, Ill.) or RC DC Protein Assay kit (Bio-Rad, Hercules, Calif.).

Immunization Schedule and Mouse Vaginal Infection Model.

Groups of 8 female mice between 7 and 9 weeks old were immunized i.vag. with gonococcal OMV (40 μg protein) of various strains as described, plus IL-12/ms (1 μg IL-12) or blank ms in a total volume of 40 μl PBS; control groups were sham-immunized with IL-12/ms or with blank ms alone. Mice were immunized 1 to 3 times with a 7-14 day interval, as indicated. After a further 2 weeks to 6 months, immunized mice were infected with 5×10⁶ CFU live N. gonorrhoeae as previously described (Jerse, Infect. Immun. 67, 5699-5708 (1999; Liu et al., J. Infect. Dis. 208, 1821-1829 (2013)), with the modification that 0.5 mg Premarin (Pfizer, Philadelphia, Pa.) was used as estradiol administered s.c. on days −2, 0, and 2. Vaginal swabs collected daily were quantitatively cultured on GC agar supplemented with hemoglobin, ISOVITALEX® (an enrichment medium) and selective antibiotics (vancomycin, streptomycin, nisin, colistin, and trimethoprim) to determine the bacterial colonization loads. The limit of detection was 100 colony-forming units (CFU) recovered per mouse. Gonococcal recovery was counted by an individual who was “blinded” to the experimental treatments, and all experiments were repeated 2 or 3 times for verification.

Assay of Serum and Mucosal Antibodies.

Samples of vaginal wash and serum were collected from the mice at the indicated time points (Liu et al., mBio 2: (2011). Gonococcus-specific IgA, IgG, IgM, and IgG subclass antibodies IgG1, IgG2a, IgG2b, and IgG3 in vaginal washes and sera were measured by ELISA on plates coated with whole gonococci, using undiluted vaginal wash and 10-fold diluted serum as starting dilutions.^{17, 26} Total IgA, IgG, and IgM concentrations in secretions were assayed by ELISA on plates coated with anti-IgA, -IgG, or -IgM antibodies (Southern Biotech, Birmingham, Ala.). H5 mouse monoclonal antibody (specific for N. gonorrhoeae porin serovar PIB3) or affinity-purified mouse IgA, IgG, and IgM (Southern Biotech) were used to establish standard curves. Bound antibodies were detected by alkaline phosphatase-conjugated goat anti-mouse IgA, IgG, IgM, IgG1, IgG2a, IgG2b, or IgG3 antibody (Southern Biotech) and p-nitrophenylphosphate substrate (Southern Biotech). Plates were read in a VersaMax microplate reader with SoftMax software (Molecular Devices, Sunnyvale, Calif.) or an ELx800 Universal microplate reader with KC Junior software (Bio-Tek Instruments, Winooski, Vt.). Antibody data were expressed as relative (fold increase) to the antibody levels detected in control samples (from sham-immunized mice) assayed simultaneously.

Flow Cytometry.

Isolated cells were washed with staining buffer twice, then incubated with the indicated antibodies for 30 min on ice, washed, and analyzed on a FACSCalibur cytometer. For intracellular staining, cells were first fixed with CYTOFIX/CYTOPERM® (eBioscience). Antibodies to mouse CD4, CD8, IFNγ, IL-4, and IL-17A conjugated with FITC, PE, or allophycocyanin were purchased from eBioscience.

Lymphocyte Isolation and Culture.

Mononuclear cells were isolated from aseptically harvested ILN using Histopaque 1083 (Sigma-Aldrich, St Louis, Mo.) density gradient centrifugation and pooled from 2 or 3 mice to provide sufficient numbers of cells for culture. CD4⁺ T cells were purified through negative selection using a Dynal CD4 cell isolation kit (Invitrogen, Carlsbad, Calif.). Cells were cultured in 24-well culture plates at a density of 2×10⁶ cells/ml in the presence of equal numbers of mitomycin C-inactivated spleen cells to serve as APC, either with no stimulus or with 2×10⁷ N. gonorrhoeae cells.

Proliferation Assays.

Cells were labeled with carboxymethyl fluorescein succinimide ester (CFSE; Sigma-Aldrich). CFSE-labeled cells were then washed twice in PBS, recounted, and stimulated as described above. Cultured cells were harvested and then stained with allophycocyanin-conjugated anti-mouse CD4 antibody. The data were acquired by gating on the CD4⁺ cell populations in a FACSCalibur cytometer. The attenuation of CFSE fluorescence was used to measure cell proliferation.

Cytokine ELISA.

IFNγ, IL-4, and IL-17A levels were measured in triplicate using ELISA kits purchased from eBioscience.

Real-Time RT-PCR.

Total cellular RNA of whole vaginas harvested from the mice was isolated with RNEASY® RNA purification Mini Kits (Qiagen, Valencia, Calif.), and was transcribed to cDNA using the ISCRIPT™ cDNA synthesis kit (Bio-Rad, Hercules, Calif.). Real-time RT-PCR was performed on an ICYCLER IQ® detection system (Bio-Rad) using SYBR® Green Dye (Bio-Rad) for real-time monitoring of the PCR. The primers used were as follows: IFNγ, 5′-TACTGCCACGGCACAGTCATTGAA-3′(SEQ ID NO: 1), 5′-GCAGCGACTCCTTTTCCGCTTCCT-3′ (SEQ ID NO: 2); IL-4, 5′-GAAGCCCTACAGACGAGCTCA-3′(SEQ ID NO: 3), 5′-ACAGGAGAAGGGACGCCAT-3′ (SEQ ID NO: 4); IL-17A, 5′-TCAGGGTCGAGAAGATGCTG-3′ (SEQ ID NO: 5), 5′-TTTTCATTGTGGAGGGCAGA-3′ (SEQ ID NO: 6); β-actin, 5′-CCTAAGGCCAACCGTGAAAAG-3′ (SEQ ID NO: 7), 5′-GAGGCATACAGGGACAGCACA-3′ (SEQ ID NO: 8). Relative quantification of target genes was analyzed based on the threshold cycle (Ct) determined by Bio-Rad IQ™ 5 optical system software.

Western Blot.

N. gonorrhoeae OMV preparations were boiled for 5 min in sodium dodecyl sulfate (SDS) loading buffer containing 2-mercaptoethanol. Protein quantification was done with the RC DC Protein Assay kit. Ten micrograms of protein from each sample was separated on 10% polyacrylamide SDS electrophoresis gels. Protein bands were transferred onto nitrocellulose membranes using the electrophoresis transfer system (Bio-Rad, Hercules, Calif., USA). The nitrocellulose membranes were blocked with PBS containing 3% skim milk overnight at 4° C. before incubation for 2 h with serum samples diluted 1:200, or vaginal wash samples diluted 1:20 in PBS containing 3% skim milk. Specific antibodies bound to N. gonorrhoeae OMV preparations were detected with horseradish peroxidase-conjugated goat anti-mouse-IgG (Santa Cruz Biotechnology, Paso Robles, Calif.) at a dilution of 1:4000. The Pierce detection kit was used for chemiluminescent detection and images were collected with a ChemiDoc MP imaging system (Bio-Rad).

Immunoproteomics.

Protein concentration in OMV was measured using DC Protein Assay Kit (Bio-Rad). Samples of OMV [300 μg and 50 μg of protein, for two dimensional (2D) SDS-PAGE-MS/MS analysis and immunoblotting, respectively] were precipitated overnight in 90% acetone, washed twice with 100% ice-cold acetone and air-dried. Protein pellets were reconstituted in 2004, of rehydration buffer (7M urea, 2M thiourea, 2% CHAPS, 2% ASB-14, 1% DTT, 2 mM TBP, 2% 3-10 IPG buffer, trace of Orange G) and used to rehydrate pH 4-7 ReadyStrip IPG strips (Bio-Rad) overnight at 25° C. Isoelectric focusing was carried out using the PROTEAN® i12™ IEF System (Bio-Rad) for a total of 26,000Vh with the following settings: 50 μA current limit, 8000V rapid ramp for 26,000Vh, 750V hold. The second dimension (2D) SDS-PAGE was performed using Criterion TGX Any kD gels (Bio-Rad). The proteins were stained overnight in Flamingo fluorescent stain (Bio-Rad) and the spots were visualized using the ChemiDoc Imaging System (Bio-Rad). For immunoblotting, separated proteins were transferred onto PVDF membranes using the TurboBlott transfer system (Bio-Rad). The membranes were blocked for 2 h in 5% milk in PBS Tween, and probed by overnight incubation with sera from immunized mice, followed by incubation with anti-mouse HRP-conjugated antibodies (Bio-Rad). Spots were visualized using Clarity Western ECL Substrate and ChemiDoc MP Imaging System (Bio-Rad). Proteins on membranes were stained with Novex Reversible Membrane Protein Stain (InVitrogen) to overlay positions of selected “anchor” spots with the Flamingo-stained 2D gels. Matching spots were excised and the proteins were trypsin digested. Samples containing extracted peptides were desalted using ZipTip C₁₈ (Millipore, Billerica, Mass.) and eluted with 70% acetonitrile/0.1% TFA, and dried in a speed vac. Desalted peptides were brought up in 2% acetonitrile in 0.1% formic acid (20 μL) and analyzed (2 μL) by LC/ESI MS/MS with a Thermo Scientific Easy-nLC II (Thermo Scientific, Waltham, Mass.) nano HPLC system coupled to a hybrid Orbitrap Elite ETD (Thermo Scientific) mass spectrometer. In-line de-salting was accomplished using a reversed-phase trap column (100 μm×20 mm) packed with Magic C₁₈AQ (5-μm 200 Å resin; Michrom Bioresources, Auburn, Calif.) followed by peptide separations on a reversed-phase column (75 μm×250 mm) packed with Magic C₁₈AQ (5-μm 100 Å resin; Michrom Bioresources) directly mounted on the electrospray ion source. A 30-minute gradient from 7% to 35% acetonitrile in 0.1% formic acid at a flow rate of 400 nL/min was used for chromatographic separations. The heated capillary temperature was set to 300° C. and a spray voltage of 2750V was applied to the electrospray tip. The Orbitrap Elite instrument was operated in the data-dependent mode, switching automatically between MS survey scans in the Orbitrap (AGC target value 1,000,000, resolution 240,000, and injection time 250 milliseconds) with MS/MS spectra acquisition in the linear ion trap (AGC target value of 10,000, and injection time 100 msec). The 20 most intense ions from the Fourier-transform (FT) full scan were selected for fragmentation in the linear trap by collision-induced dissociation with normalized collision energy of 35%. Selected ions were dynamically excluded for 15 sec with a list size of 500 and exclusion mass by mass width ±0.5. Data analysis was performed using Proteome Discoverer 1.4 (Thermo Scientific). All identified peptides were searched against a N. gonorrhoeae database (FA1090, FA19, and MS11) combined with cRAP.fasta, a database of common contaminants (thegpm.org/crap/); this creates a list of proteins commonly found in proteomics experiments that are present by accident or unavoidable contamination. Trypsin was set as the enzyme with maximum missed cleavages set to 2. The precursor ion tolerance was set to 10 ppm and the fragment ion tolerance was set to 0.8 Da. Variable modifications included oxidation on methionine (+15.995 Da) and carbamidomethyl on cysteine (+57.021 Da). Data were searched using Sequest HT. All search results were run through Percolator for scoring

Statistical Analysis.

Data are expressed as the mean±standard error of the mean (SEM). Data on the effect of immunization on recovery of N. gonorrhoeae after inoculation were analyzed using two-way ANOVA for repeated measures with Fisher's protected least significant difference post-hoc tests. In addition, Kaplan-Meier analysis with log-rank tests was used to compare clearance of infection (defined as the first of 3 successive days of zero recovery) between treatment groups. For immune response data, unpaired two-tailed t tests were used to compare the mean values between two groups, or ANOVA with Bonferroni post-hoc tests was used for multiple comparisons. P<0.05 was considered statistically significant. Statistical analyses were performed using Microsoft Excel or Prism 5 (GraphPad Software, San Diego, Calif.).

### Example 4

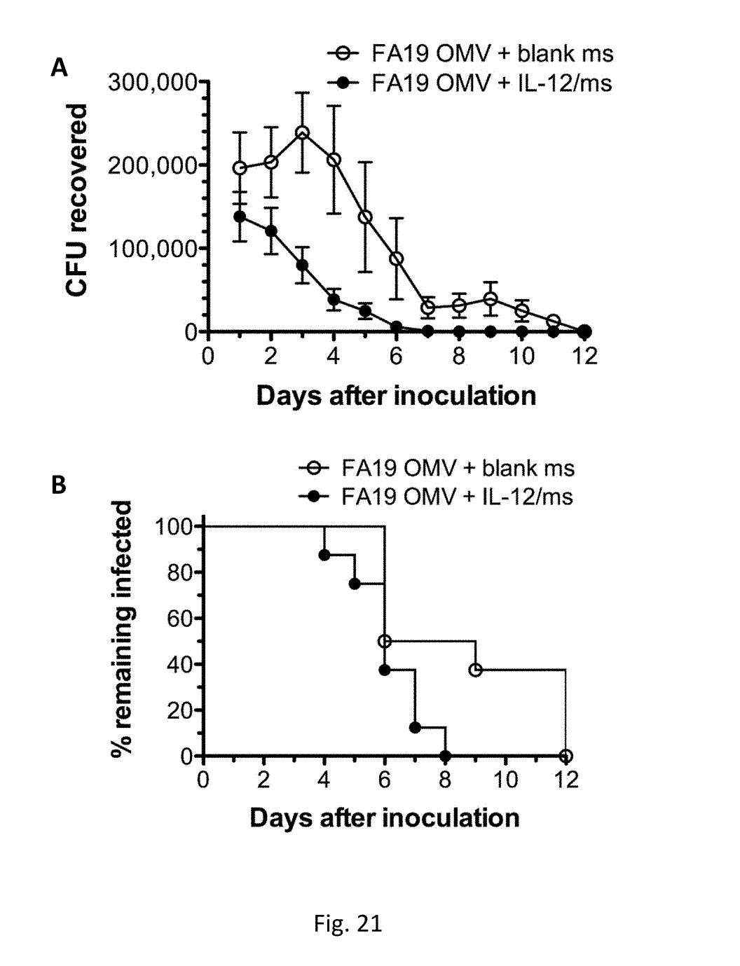

This examples describes the administration of OMVs and IL-12 ms intranasally. Female mice (8 per group) were immunized intranasally with OMV (40 μg protein, strain FA19) plus IL-12/ms (1 μg IL-12) or blank ms on days 0 and 14. Two weeks later all mice were challenged intravaginally with 5×10⁶ CFU of N. gonorrhoeae strain FA1090. Vaginal swabs collected daily were diluted and cultured quantitatively on GC agar plates containing selective antibiotics. Results show that mice immunized with OMVs plus IL-12 ms cleared the infection significantly faster than the control groups (p<0.01, FIG. 21). In addition, the bacterial colonization loads were significantly lower in the mice immunized with OMVs plus IL-12 ms.

### Example 5

Studies were carried out to further illustrate protective effect of compositions comprising OMV and IL-12 microspheres delivered via the intranasal route and comparison of the intranasal route with the intravaginal route.

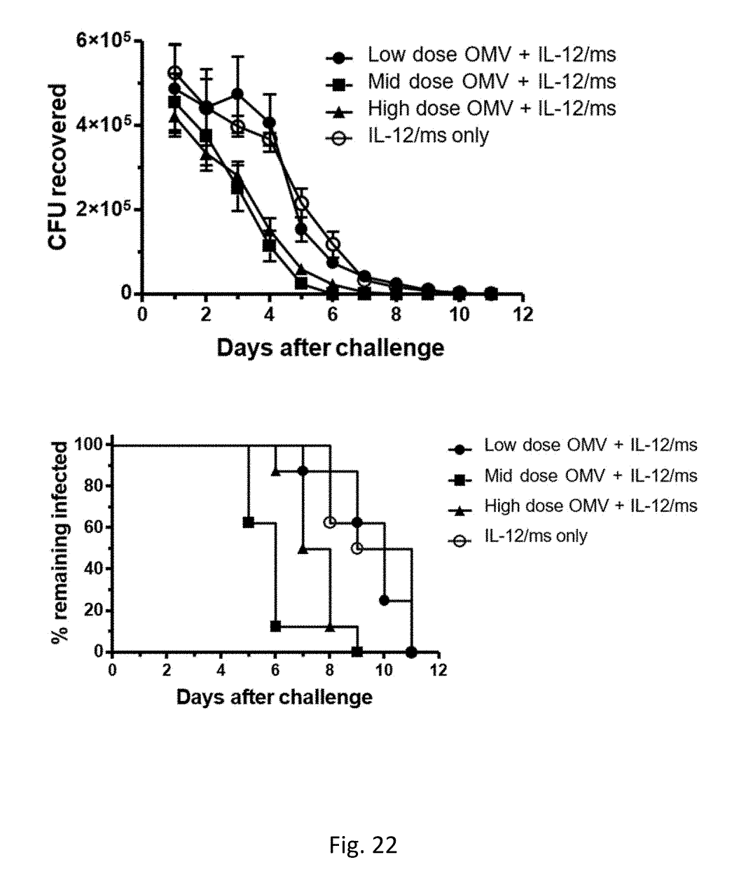

Mice were immunized intranasally (i.n.) with different doses of OMV prepared from N. gonorrhoeae strain FA1090: 15 μg, 30 μg, or 60 μg of OMV protein, together with microencapsulated IL-12 (IL-12/ms; 1 μg of IL-12), or with IL-12/ms alone. Immunizations were repeated 2 weeks later. Two weeks after the last dose, mice were challenged intravaginally with live N. gonorrhoeae strain FA19, and the course of infection was followed by daily vaginal swabbing and plating as described previously. As shown in FIG. 22, mice immunized with the low dose of 15 μg OMV did not show accelerated clearance of the infection compared to the control group immunized with IL-12/ms alone (median clearance times: 10 days for both groups). Mice immunized with the usual (mid) dose 30 μg or with an increased dose of 60 μg OMV cleared the infection significantly faster (median clearance times 6 days and 7.5 days, respectively) than the control group (P<0.001, Kaplan-Meier, log-rank test).

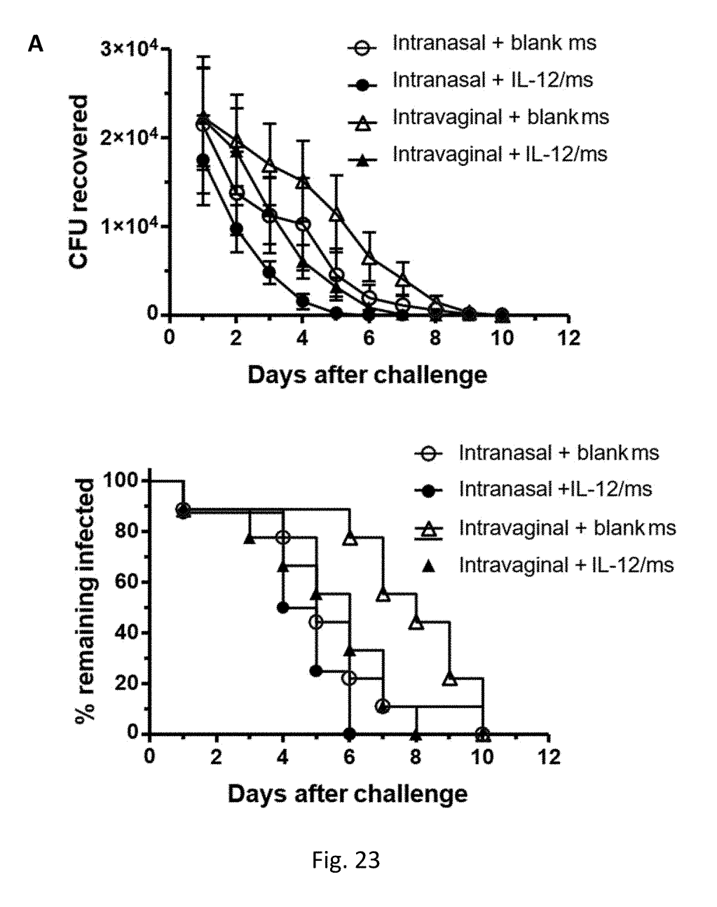

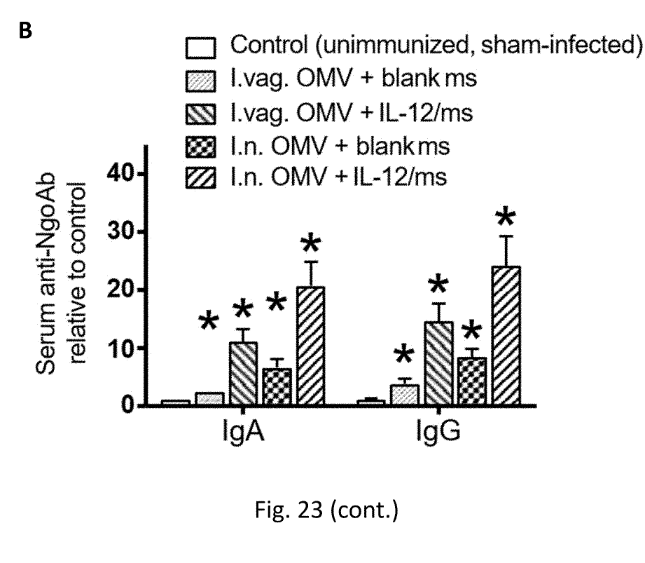

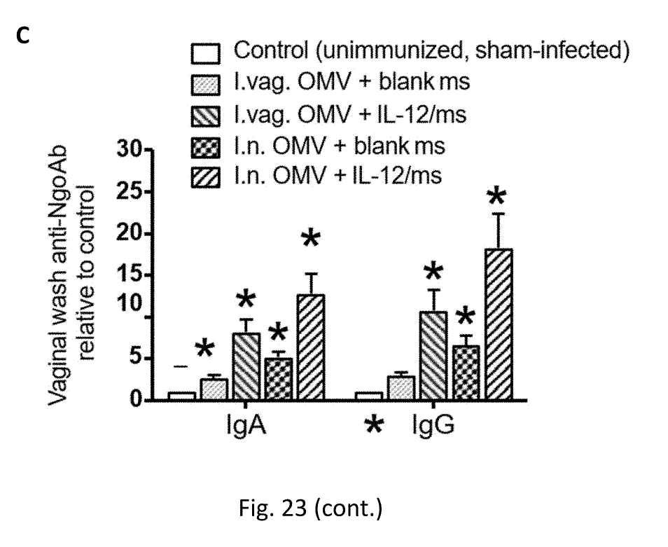

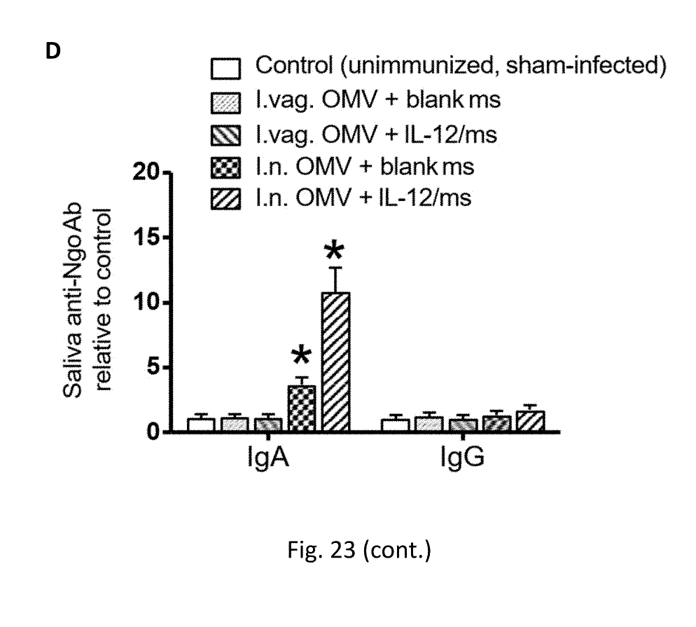

Intranasal (i.n.) immunization was compared with intravaginal (i.vag.) immunization as follows. Mice were immunized with 30 μg of gonococcal FA1090 OMV (as protein) plus IL-12/ms (1 μg IL12) or blank microspheres (ms) either i.n. or i.vag. and immunizations were repeated after 2 weeks. Two weeks later, mice were challenged i.vag. with live N. gonorrhoeae strain FA19 (heterologous challenge), and the course of infection was followed by daily vaginal swabbing and plating as described previously. As shown in FIG. 23A-D, mice immunized with OMV plus IL-12/ms i.n. cleared the infection comparably to mice immunized i.vag. After clearance of the infections (2 weeks after challenge) samples of serum, vaginal wash, and saliva were collected for assay of IgG and IgA anti-gonococcal antibodies by ELISA. Mice immunized i.n. with IL-12/ms cleared the infection comparably to those immunized i.vag. with the same vaccine (FIG. 23A). Mice immunized i.n. with OMV plus IL-12/ms developed IgG and IgA antibodies in serum (FIG. 23B) and vaginal wash (FIG. 23C) at levels higher than those immunized i.vag. Mice immunized i.n. with OMV plus IL-12/ms also developed high levels of salivary IgA antibodies (FIG. 23D).

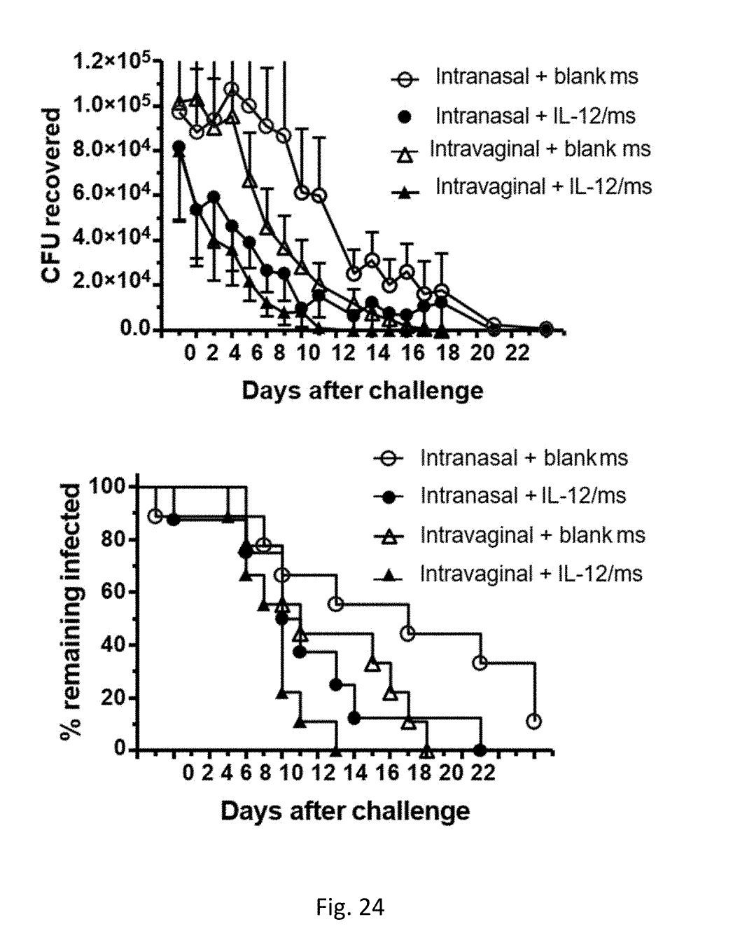

When mice were challenged with the same strain as used for the preparation of the OMV, FA1090 (homologous challenge), mice immunized with OMV plus IL-12/ms i.n. (as described for FIG. 3), cleared the infection comparably to those immunized i.vag. (FIG. 24).

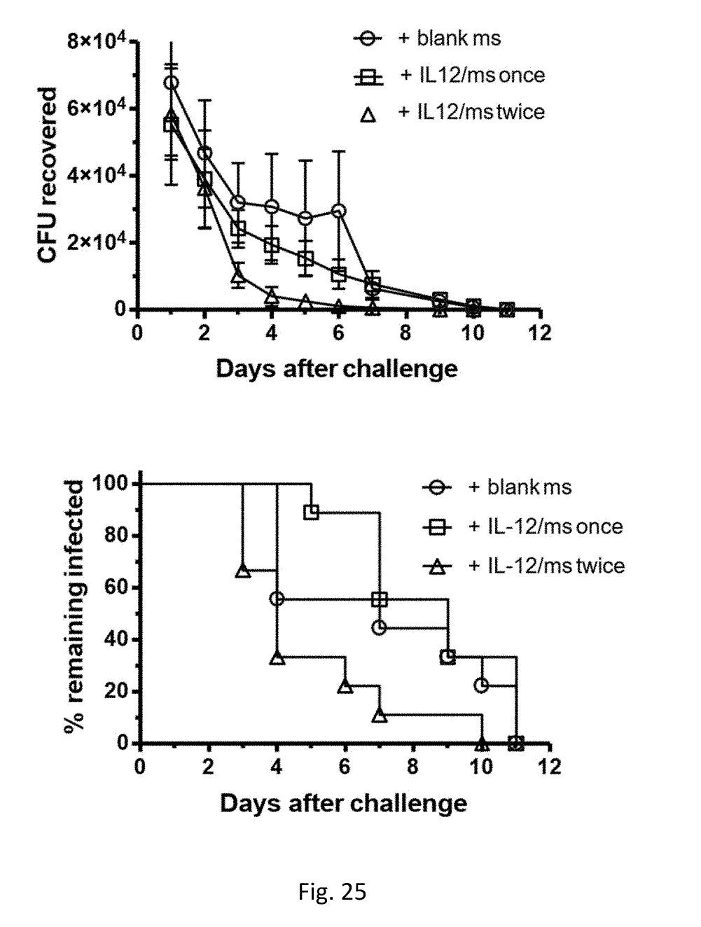

Mice were immunized i.n. once or twice with gonococcal FA1090 OMV (30 μg protein) plus IL-12/ms (1 μg IL-12); control mice received blank ms. Two weeks after the second immunization, mice were challenged i.vag. with N. gonorrhoeae MS11 (heterologous challenge). As shown in FIG. 25, mice immunized twice with OMV plus IL-12/ms cleared the infection faster (median clearance time: 4 days) than those immunized with one dose (median clearance time 9 days) or than control mice (median clearance time 7 days); P=0.0155 (Kaplan-Meier analysis, log-rank test).

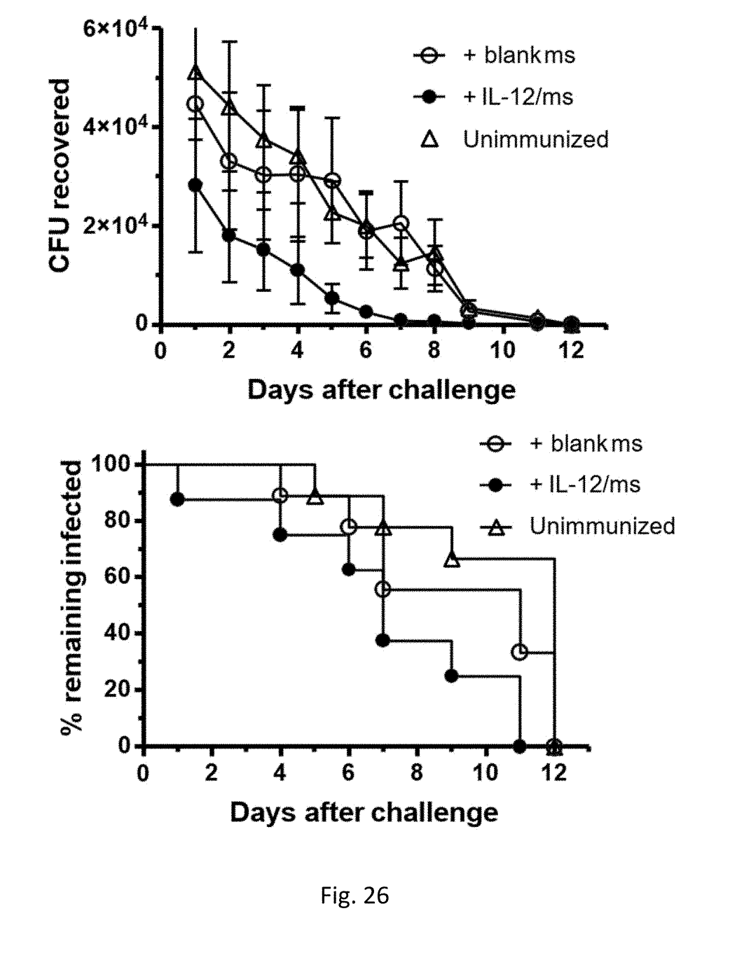

Mice were immunized i.n. with OMV (30 μg protein) prepared from N. gonorrhoeae MS11, plus either IL-12/ms (1 μg IL-12) or blank ms, and challenged with heterologous gonococcal strain FA1090. As shown in FIG. 26, mice immunized with MS11 OMV plus IL-12/ms cleared the infection faster (median clearance time: 7 days) compared to those immunized with MS11 OMV plus blank ms (median clearance times: 11 days), or compared to unimmunized control mice (median clearance time: 12 days); P=0.0231 (Kaplan-Meier analysis, log-rank test).

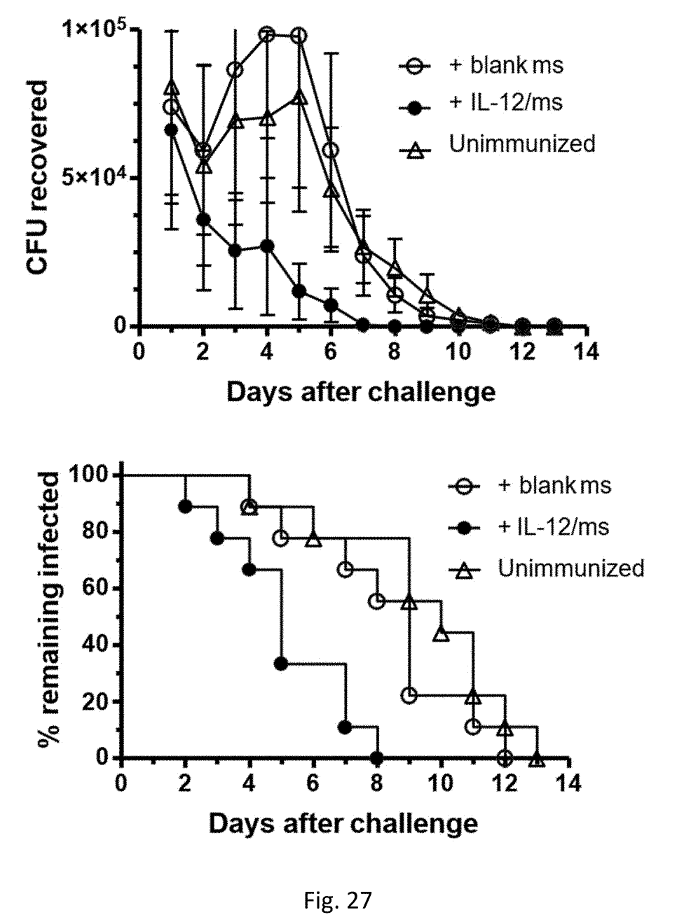

Mice were immunized i.n. with OMV (30 μg protein) prepared from N. gonorrhoeae MS11, plus either IL-12/ms (1 μg IL-12) or blank ms, and challenged with heterologous gonococcal strain FA19. As shown in FIG. 27, mice immunized with MS11 OMV plus IL-12/ms cleared the infection faster (median clearance time: 5 days) compared to those immunized with MS11 OMV plus blank ms (median clearance time: 9 days), or compared to unimmunized control mice (median clearance time: 10 days). P=0.0007 (Kaplan-Meier analysis, log-rank test)

It should be noted that the strains of N. gonorrhoeae used in these experiments express different porin types. Gonococcal porin PorB is the major outer membrane protein in N. gonorrhoeae, and is represented by two major types, PorB-1A and PorB-1B, each having many sub-types. Strain FA1090 has porin type PorB-1B, strain MS11 also has porin type PorB-1B although a different sub-type from FA1090, and strain FA19 has porin type PorB-1A. In addition, these strains express different Opa proteins and lipooligosaccharide structures, thus they represent widely different antigenic variants of N. gonorrhoeae.

Although the present invention has been described with respect to one or more particular embodiments, it will be understood that other embodiments of the present invention may be made without departing from the spirit and scope of the present invention.

What is claimed is:

1. A method for reducing the risk of a genital tract infection of N. gonorrhoeae in an individual comprising the steps of administering to the individual intranasally an amount of IL-12 incorporated in polymeric microspheres and outer membrane vesicles (OMVs) from N. gonorrhoeae effective to reduce the risk of contracting the genital tract infection.

2. The method of claim 1, wherein the IL-12 microspheres and the OMVs are delivered in the same composition.

3. The method of claim 1, wherein the IL-12 microspheres and the OMVs are administered multiple times over a period of up to three weeks.

4. The method of claim 3, wherein the IL-12 microspheres and the OMVs are administered from 2 to 4 times with an interval of about at least 1 week in between the administrations.

5. The method of claim 4, wherein the IL-12 microspheres and the OMVs are administered twice with an interval of about 2 weeks in between the two administrations.

6. The method of claim 1, wherein the OMVs are in the range of 0.01 to 2000 μg protein per dose.

7. The method of claim 6, wherein the OMVs are in the range of 0.01 to 1000 μg protein per dose.

8. The method of claim 1, wherein the OMVs are in the range of 0.5 to 30 μg/kg body weight.

9. The method of claim 1, wherein the IL-12 is in the range of 1 to 500 μg per dose.

10. The method of claim 1, wherein the IL-12 is in the range of 10 ng to 10 μg per kg body weight.

11. The method of claim 1, wherein the OMVs are in the range of 0.01 to 2000 protein per dose and IL-12 is in the range of 1 to 500 μg per dose.

12. A composition for intranasal administration comprising N. gonorrhoeae outer membrane vesicles (OMV) and IL-12, wherein the IL-12 is incorporated in polymeric microspheres, in a pharmaceutical carrier.

13. The composition of claim 12, wherein OMVs are in the range of 0.01 to 2000 protein per dose and IL-12 is in the range of 1 to 500 μg per dose.

14. The composition of claim 12, wherein the composition is substantially free of soluble IL-12.

15. A kit for intranasally vaccinating an individual against N. gonorrhoeae infection comprising multiple doses of a composition suitable for intranasal administration comprising N. gonorrhoeae outer membrane vesicles (OMV) and IL-12, wherein the IL-12 is incorporated in polymeric microspheres, in a pharmaceutical carrier, and wherein the amount of OMVs per dose is 0.01 to 2000 μg protein and IL-12 is per dose is 1 to 500 μg, and optionally instructions for administration of the composition.

16. A kit for intranasally vaccinating an individual against N. gonorrhoeae infection comprising separately:
i) a composition suitable for intranasal administration comprising N. gonorrhoeae outer membrane vesicles (OMV), wherein the amount of OMVs per dose is 0.01 to 2,000 μg;
ii) a composition suitable for intranasal administration comprising IL-12, wherein the IL-12 is incorporated in polymeric microspheres, and the amount of IL-12 per dose is 1 to 500 μg; and
iii) optionally instructions for combining i) and ii) and administration of the combined composition.


SEQUENCE LISTING (8 sequences)

SEQ ID NO: 1 - DNA, 24 bp, Artificial Sequence
  Primer
tactgccacggcacagtcattgaa

SEQ ID NO: 2 - DNA, 24 bp, Artificial Sequence
  Primer
gcagcgactccttttccgcttcct

SEQ ID NO: 3 - DNA, 21 bp, Artificial Sequence
  Primer
gaagccctacagacgagctca

SEQ ID NO: 4 - DNA, 19 bp, Artificial Sequence
  Primer
acaggagaagggacgccat

SEQ ID NO: 5 - DNA, 20 bp, Artificial Sequence
  Primer
tcagggtcgagaagatgctg

SEQ ID NO: 6 - DNA, 20 bp, Artificial Sequence
  Primer
ttttcattgtggagggcaga

SEQ ID NO: 7 - DNA, 21 bp, Artificial Sequence
  Primer
cctaaggccaaccgtgaaaag

SEQ ID NO: 8 - DNA, 21 bp, Artificial Sequence
  Primer
gaggcatacagggacagcaca